(19) United States
(12) Patent Application Publication
(10) Pub. No.: US 2021/0290109 A1
(43) Pub. Date: Sep. 23, 2021

(54) RANGE OF MOTION SYSTEM, AND METHOD

(71) Applicant: Virtusense Technologies, LLC, Peoria, IL (US)

(72) Inventors: Deepak Gaddipati, Peoria, IL (US); Troy Earley, Peoria, IL (US)

(21) Appl. No.: 17/220,235

(22) Filed: Apr. 1, 2021

Related U.S. Application Data
(62) Division of application No. 14/776,362, filed on Sep. 14, 2015.
(60) Provisional application No. 61/778,763, filed on Mar. 13, 2013.

(51) Int. Cl. A61B 5/11; A61B 5/00

(52) U.S. Cl. CPC A61B 5/1128; A61B 5/4023; A61B 5/1113; A61B 5/112; A61B 5/1117; A61B 5/0013; A61B 2560/0475; A61B 5/0022; A61B 5/0077; A61B 5/1114; A61B 5/1121; A61B 5/4824; A61B 5/002

(57) ABSTRACT

A system and method for range of motion evaluation and recording for physical therapy, ergonomics, training, and individual rehabilitation. While talking with the physical therapists, a frequently mentioned problem they encountered was to evaluate the range of motion of a patient during the performance of functional movements like standing up from a chair, taking objects off of the ground, squatting, and gait analysis. Physical therapists currently use a goniometer to measure the motion of a single angle and visually inspect for inconsistencies and subjectively assess the patient during performance of functional tasks.

### FIELD

A range of motion evaluation (ROME) and recording system, and method for physical therapy. ergonomics, training, industrial rehabilitation, medical simulation, task efficiency measurement, assembly/manufacturing workflow analysis, GAIT analysis, and diagnosis.

### BACKGROUND

While talking with the physical therapists, a frequently mentioned problem they encountered was to evaluate the range of motion of a patient during the performance of functional movements like standing up from a chair, taking objects off of the ground, squatting, and gait analysis. Physical therapists currently use a goniometer to measure the motion of a single angle and visually inspect for inconsistencies and subjectively assess the patient during performance of functional tasks.

Manufacturing companies lose significant amounts of money due to work related injuries each year. Companies are proactively taking two steps to reduce the incidents for accidents, including 1) preventative care through ergonomics, and training; and 2) getting injured worker back to work through industrial rehabilitation.

Current preventative care includes an ergonomist observing the workers'₁ actions to improve the designs for tools, platforms and actions to reduce the strain on the workers body. This analysis is subjectively performed by the ergonomist, as it is prohibitively expensive, difficult and time consuming to collect objective data on workers joint movement while the worker is performing a functional task.

Workers recovering from injuries are often sent to industrial rehab programs. These programs measure the capabilities of an injured worker in a mockup factory setting and evaluate, in person, their performance on certain core tasks using standards like NIOSH and OSHA. It takes a lot of time to perform this evaluation and industrial rehab evaluator should monitor each action and take measurements with tape and goniometer's to evaluate the worker's ability to perform each task. ROME can be used to automatically perform these actions and comply with NIOSH and OSHA standards to calculate the workers rehab metrics.

Manufacturing companies can lose significant amounts of money to work related injuries each year. These companies manage work related injuries by providing preventative care through ergonomics, training, and getting injured workers back to work through industrial rehabilitation.

Again, current preventative care practices include using an ergonomist to observe the workers' actions to improve the designs for tools, platforms and actions to reduce the strain on the workers body. This analysis is subjectively performed by the ergonomist and can be prohibitively expensive, difficult and time consuming as it involves collecting objective data on workers' joint movement while performing a functional task. Furthermore, industrial rehabilitation programs generally measure the capabilities of an injured worker in a mockup factory setting and evaluate, in person, their performance on certain core tasks using standards like NIOSH and OSHA.

This evaluation is time consuming, particularly where the evaluator should monitor each action and take measurements with tape and goniometer to evaluate the worker's ability to perform each task. In addition, evaluating the range of motion of a patient during the performance of functional movements like standing up from a chair, taking objects off of the ground, squatting and gait analysis can be challenging, as physical therapists currently use a goniometer to measure the motion of a single angle and visually inspect for inconsistencies and subjectively assess the patient during performance of functional tasks.

### SUMMARY

A range of motion evaluation system comprising a motion-sensing device for tracking and monitoring a person's motion.

A range of motion evaluation system comprising a motion-sensing device for tracking and monitoring a person's motion, the motion-sensing device comprising a projector and camera.

A range of motion evaluation system comprising a motion-sensing device for tracking and monitoring a person's motion, the motion-sensing device comprising a projector and camera, wherein the projector emits a known infrared pattern and the camera captures infrared points in the scene.

A range of motion evaluation method comprising tracking the movement of multiple joints of an individual in three dimension; and generating real-time metrics for evaluating the individual's movements.

A range of motion evaluation method comprising tracking the movement of multiple joints of an individual in three dimension; generating real-time metrics for evaluating the person's movements; and training the person to perform the movement in a safer manner.

A range of motion evaluation method comprising tracking the movement of multiple joints of an individual in a three-dimensional scene; emitting a known infrared pattern into the scene; capturing infrared points in the scene; and converting the infrared pattern into a detailed depth map of the scene.

A range of motion evaluation method comprising tracking the movement of multiple joints of an individual in a three-dimensional scene; emitting a known infrared pattern into the scene; capturing infrared points in the scene; converting the infrared pattern into a detailed depth map of the scene; and tracking specific points on an individual.

A range of motion evaluation method comprising tracking the movement of multiple joints of an individual in a three-dimensional scene; emitting a known infrared pattern into the scene; capturing infrared points in the scene; converting the infrared pattern into a detailed depth map of the scene; and tracking relative motions of various joints of the individual to provide real-time data for angular movements, velocity, and acceleration.

ROME (Range of Motion Evaluation) can track twenty (20) different joints on the patient using markerless IR based depth mapping technology. The ROME platform uses an available motion tracking device to track and monitor what the observed person does. This sensor combines a projector and camera system to estimate depth from the camera. The projector emits a known infrared pattern and the camera to capture the infrared points in the scene. Image processing algorithms are used to convert this infrared pattern into a detailed depth map of the scene from the camera's point of view. The depth map is further processed with the sensors custom software development kit to identify and track specific points on individuals in front of the camera. This data is used by ROME to monitor actions of the tracked people, and determine what and how actions are being performed. Using this sensor, ROME can track the relative motions of various joints to provide real-time data for angular movements, velocity and acceleration in an automated fashion.

The applications for ROME for industrial, physical therapy, and ergonomics, include:

1) Ergonomics and Safety Assessment

2) Ergonomic Task Training

3) Task Efficiency measurement

4) Assembly/Manufacturing Work flow analysis

5) Telemetric Ergonomic Assessment and Training

6) Physical therapy

7) GAIT analysis

8) Measuring balance with modified Clinical Test for Sensory Interaction on Balance (CTSIB), and functional tests

9) Assessing fall risks of inpatient in hospital/nursing home

10) Monitoring Activities of Daily Living (ADL)

ROME can provide data to be used for an ergonomics and safety assessment of workers performing their jobs. This can be used by ergonomics personnel to verify that a worker is performing a task in the most ergonomically efficient manner, and decide if training is needed or not. ROME can be used as an ergonomic task trainer system to provide training to workers on the proper way to ergonomically perform a manufacturing or assembly task. This would provide the worker feedback and suggestions on how to modify their performance to be better for their long term health, as well as document the workers training and progress for the companies records.

Before workers are hired or after recovering from an injury, they are sent to an industrial rehab center to perform tests to prove to insurance companies that they are fit for work. The ROME system can capture this information to determine how effective a worker they could be. This ROME system could be used for work flow analysis by being placed in the manufacturing plant to monitor the effectiveness of workers on an active assembly line. This could provide information about quality of performance and potential line disruptions to management before major issues arise.

The last application is a remote system that can provide all of this ergonomic evaluation and training benefits to small manufacturing plants. This would allow the all the manufacturing workers in large companies to be trained to the same standards, regardless how large or remote of a plant they work in. Currently, corporations hire and fly ergonomists to work sites to perform an evaluation and assessment, and suggest training for the workers. Usually when the ergonomist leaves, the workers return to their normal practices and continue to develop avoidable injuries. This remotely connected ROME system can transmit all the information to a centralized point, which can allow the ergonomists to perform their jobs better with substantially lower travel costs.

ROME's markerless technology can objectively track the movement of multiple joints in three dimensions to generate real-time metrics that can be used by ergonomists to evaluate and train the employees to perform a job in a safe way.

The range of motion evaluation (ROME) system that can be used to track twenty different joints on the patient using markerless infrared (IR) based depth mapping technology. ROME's markerless technology can objectively track the movement of multiple joints in three (3) dimensions to generate real-time metrics that can be used by ergonomists to evaluate and train the employees to perform a job in a safe way. ROME can also be used to automatically perform these actions and comply with NIOSH and OSHA standards to calculate the workers rehabilitation metrics.

The ROME platform uses an available motion-tracking device (i.e. sensor) to track and monitor what the observed person does. This sensor combines a projector and camera system to estimate depth from the camera. The projector emits a known infrared pattern and the camera to capture the infrared points in the scene. Image processing algorithms are used to convert this infrared pattern into a detailed depth map of the scene from the camera's point of view. The depth map is further processed with the sensors custom software development tool or kit to identify and track specific points on individuals in front of the camera. This data is used by ROME to monitor actions of the tracked people, and determine what and how actions are being performed. Using this sensor, ROME can track the relative motions of various joints to provide real-time data for angular movements, velocity and acceleration in an automated fashion.

Industrial, physical therapy, and ergonomics applications of ROME include use for ergonomics and safety assessment, ergonomic task training, task efficiency measurement, assembly/manufacturing workflow analysis, GAIT analysis and telemetric ergonomic assessment and training.

ROME can provide data to be used for an ergonomics and safety assessment of workers performing their jobs. This can be used by ergonomics personnel to verify a worker is performing a task in the most ergonomically efficient manner and decide if training is needed. In addition, ROME can be used as an ergonomic task trainer system to provide training to workers on the proper way to ergonomically perform a manufacturing or assembly task. This would provide the worker feedback and suggestions on how to modify their performance to be better for their long-term health, as well as document the workers training and progress for the companies' records.

ROME can be used for task efficiency measurement, as well as assembly and/or manufacturing workflow analysis. Before workers are hired, or after recovering from an injury, they are sent to an industrial rehabilitation center to perform tests to prove to insurance companies that they are fit for work. The ROME system can capture this information to determine how effective a worker they could be. The ROME system could be used for workflow analysis by placement in a manufacturing plant to monitor the effectiveness of workers on an active assembly line. This could provide information about quality of performance and potential line disruptions to management before major issues arise.

The ROME system can provide ergonomic evaluation and training benefits to small manufacturing plants remotely allowing manufacturing workers in large companies to be trained to the same standards, regardless how large or remote of a plant they work in. Currently, corporations hire and fly ergonomists to work sites to perform an evaluation and assessment, and suggest training for the workers. Usually when the ergonomist leaves, the workers return to their normal practices and continue to develop avoidable injuries. The remotely connected ROME system can transmit information to a centralized point, which allows the ergonomists to perform their jobs better with substantially lower travel costs.

ROME can be used for GAIT analysis on a wide range of patient conditions including Alzheimer's, prosthetics and orthotics, and knee and hip problems. In the current standard of care, a rehabilitation therapist visually monitors the motion of the patient and observes for inconsistencies in the movement of the hips, knees, ankles and arms while they walk, get up from a chair and/or squat. As ROME can automatically detect location of multiple joints real-time, real-life motion can be captured. When performing the GAIT analysis, the patient would be given a wireless pain indicator to report when the patient encounters pain while doing a task. This would objectify the pain levels and link them with real-time 3-dimensional motion capture and joint orientations.

### Features

ROME-Industrial Rehab and Ergonomics
1. Objective data capture for measuring interactions with machines in assembly line, warehouse and manufacturing settings;
2. 1., with marker-less sensors that do not interfere with the workers actions;
3. Create a template or multiple templates that indicate an ergonomic, safe and efficient way of performing a job;
4. Automatically derive metrics from the templates in 3 to create rule sets;
5. Evaluating multiple workers with respect to templates 3 and rule sets 4, using classifiers and generate an automatic/semi-automatic way to ergonomic metrics;
6. Generating concise evaluation reports that can be provided to improve inter-department communication and workers safety;
7. An avatar based self-training module to train the workers in an adaptive fashion on improved ways of performing the job;
8. Using collected data to document evidence of evaluations performed on worker and document improvements attained through self-training process for possible uses relating to workers compensation cases or insurance fraud;
9. Ability to perform 1. through 8. remotely at multiple locations without the physical presence of an ergonomist; and
10. Ability to record the joint movement while performing job functions in 3d and play it back in a 3d viewer. Ability to visually compare (side by side) this movement from multiple time points or across multiple people.

### PT-ROME

1. Objective data capture of joint angles exerted during a evaluation of range of motion of the patient;
2. 1., with unobtrusive marker-less sensors that do not interfere with the therapist's ability to observe;
3. A database with the recorded evaluation history for a patient at each step during treatment that is accessible during treatment and evaluation;
4. Capturing at what time during an examination the patient is experiencing pain, using a patient operated device;
5. Using the information from both 1. and 4. to generate metrics about treatment progress and patient condition;
6. Generating concise evaluation reports that can be provided to physicians, patients to improve communication and patient treatment process; and
7. Using collected data to provide evidence if a patient does or does not have a medical condition, including possible uses relating to legal cases or insurance fraud.

### PT-ROME-Gait

1. Objective data capture of joint angles exerted during the performance of a gait assessment;
2. 1., with unobtrusive marker-less sensors that do not interfere with the therapist's ability to observe;
3. A database with the recorded evaluation history for a patient at each step during treatment that is accessible during treatment and evaluation;
4. Capturing at what time and what motion during the GAIT assessment the patient is experiencing pain, using a patient operated device;
5. Using the information from both 1. and 4. to generate metrics about treatment progress and patient condition;
6. Generating concise evaluation reports that can be provided to physicians, patients to improve communication and patient treatment process; Using collected data to provide evidence if a patient does or does not have a medical condition, including possible uses relating to legal cases or insurance fraud; and
8. Collected data to assess patients with fall risk and concussions.

### PT-ROME-Balance

1. Objective data capture of center of body, sway of spine, head, upper extremity, lower extremity, and various limbs on body;
2. 1., with unobtrusive and minimal setup time for marker-less sensors that do not interfere with the therapist's ability to observe;
3. Ability to automatically detect multiple people and assign the closest person to the sensor as patient;
4. Ability to select a patient among multiple people on the screen;
5. Ability to measure total sway distance during a period of time, average sway distance, average sway velocity, peak sway velocity, average sway acceleration, peak sway acceleration, Range of sway in X, Y and Z axes;
6. 5., using any/all of the measures to compute a numeric score to indicate balance of a person;
7. Ability to separate visual, vestibular and somatosensory components of persons balance based on CTSIB measurements from 5;
8. A database with the recorded balance history for a patient at each step during treatment that is accessible during treatment and evaluation;
9. Using the information from both 6. and 7. to generate metrics about treatment progress and patient condition;
10. Generating concise evaluation reports that can be provided to physicians, patients to improve communication and patient treatment process;
11. Using collected data to provide evidence if a patient does or does not have a balance condition, including possible uses relating to legal cases or insurance fraud; and
12. Compare the patient score with collected data to assess patient's condition with respect to age groups, sex and disease conditions.

### ROME—Monitoring ADL and Assessing Fall Risks of Inpatient in Hospital/Nursing Home:

1. Objective data capture of center of body, sway of spine, head, upper extremity, low extremity, and various limbs on body
2. 1., with unobtrusive and minimal setup time for marker-less sensors that do not interfere with the healthcare providers ability to observe
3. Ability to automatically detect multiple people and assign the closest person to the sensor as patient
4. Ability to select a patient among multiple people on the screen
5. Ability to measure location of person within a room, measure position of the person with respect to the bed, chair, and floor, locate a person in multiple rooms at various periods of the day
6. Ability to automatically detect and measure the interaction time and location in single/multiple rooms with other people and breaking the time spent with each person respectively
7. Ability to automatically generate alert messages based on the position of the patient with respect to the floor and bed
8. Ability to send the automated messages to remote hospital networks and emergency providers in real-time
9. Generate live reports of ADL that can be accessed by family and healthcare providers monitoring the condition of the patient.
10. A database with the recorded ADL history for a patient, An alert service that alerts healthcare providers of abnormal behavior of the patient based on the prior history

### Advantages

### ROME-Industrial Rehab and Ergonomics

The prior art for this technology is ergonomics personnel going and physically observing the performance of a person, or a video recording system that will be monitored by a person to extract the same metrics. ROME-Industrial Rehab and Ergonomics allows for automatic data capturing and processing, and can be easily deployed in remote facilities. These factors alone cut down significant amounts of travel time and help automatically collect metrics the ergonomics personnel would collect manually. This system also captures and recorded data, which can be useful for employment records or to satisfy regulatory or legal needs. The self-training aspect of the ergonomics task trainer is also more likely to help improve a workers ergonomic health, as the training can be done cheaply and regularly.

### PT-ROME

The prior art for PT-ROME is a physical therapist or assistant that uses a goniometer to manually measure each angle and document any abnormalities during the performance of the action. PT-ROME captures this data automatically, and can provide information about how fluid a motion is and provide a recorded video of patient data for the therapist to review. Combining information about the maximum achieved angle and the intensity of pain experienced by the patient, metrics are derived to help show the progress of treatment for the therapists.

The prior art for PT-ROME-GAIT is observational based assessment by orthopedists or other doctors, or expensive joint and motion capturing setups. When a doctor performs an assessment, they perform a subjective evaluation without any recorded data on the patient. The PT-ROME-GAIT records the action of the joints simultaneously, providing objective data for diagnosis and evaluation. The motion capture setups are typically prohibitively expensive and require good lighting and often involve indicators being placed on the patient's body to track points exactly. PT-ROME-GAIT uses a low-cost tracking sensor, which uses marker-less technology to track the specific points on the person being observed.

### ROME-Balance and Functional Assessment

Physical rehabilitation professionals typically treat patients with varying levels of balance dysfunction to reduce a person's risk of falling and improve their overall function. In most cases, balance is assessed by judging the amount of postural sway of the human body and assessing a person's ability to maintain upright posture when presented with various physical challenges. Rehab professionals currently need to administer test for balance, gait on separate machines and functional tests in a subjective manner. There is no quantitative way to track the status of the patient's improvement over a period of time, as the improvement of patient is based on a combination of objective and subjective tests. Rehab professionals face unique challenges including: the ability to objectively document as to the extent and nature of balance deficit, the ability to house and employ an objective balance measurement device, the capability to document and communicate the need for specific skilled therapeutic treatments to the patient and third-party payers, and the ability to monitor the effectiveness of treatments over time.

### ROME—Monitoring ADL and Assessing Fall Risks of Inpatient in Hospital/Nursing Home:

Low-cost autonomous systems are needed to continuously monitor older adults to enable them to continue living in independent settings for longer, lowering the need for expensive retirement care facilities. These low-cost systems are needed not only to detect adverse events such as falls, but also to assess the risk of such events. People with sudden reduced physical activity need special or immediate attention, even when the patient does not recognize the reduction. Deterioration due to chronic diseases such as heart failure, diabetes, and Alzheimer's disease usually correlates with decreased activities. Detecting these early signs of distress can potentially save lives and reduce the high costs associated with emergency care. Rome for Balance can detect a senior in a track his ADL even when he is not the only person living in the house.

### BRIEF DESCRIPTION OF DRAWINGS

FIG. 1 is a diagrammatic view of a ROME for Balance, Function and GAIT.

FIG. 2 is a diagrammatic view of ROME for Ergonomics and Efficiency.

FIG. 3 is a diagrammatic view of ROME for Inpatient Fall Detection.

FIG. 4 is a diagrammatic view of track points of a person.

FIG. 5 is a diagrammatic view of a table of identification of tracked points.

FIG. 6 is diagrammatic view of an example program of ROME for Ergonomics.

FIG. 7 is a diagrammatic view of an Ergonomic Task Trainer Warning.

FIG. 8 is a diagrammatic view of a Task Efficiency Measurement Example Program.

FIG. 9 is a diagrammatic view of a ROME for Area Sterility.

FIG. 10 is a diagrammatic view of a Patient Pain Indicator.

FIG. 11 is a diagrammatic view of a ROME Evaluation Selection.

FIG. 12 is a diagrammatic view of a ROME Playback System.

FIG. 13 is a diagrammatic view of a ROME Evaluation.

FIG. 14 is a diagrammatic view of a ROME Pain Event Input.

FIG. 15 is a diagrammatic view of ROME for balance measurement marking patient with the remote.

FIG. 16 is a diagrammatic view of ROME for balance, selecting criteria for modified CTSIP test.

FIG. 17 is a diagrammatic view of tracking sway of center of the body for CTSIB test to assess balance.

FIG. 18 is a diagrammatic view of auto-detect when patient moved during CTSIB test.

FIG. 19 is a diagrammatic view of marking patient for functional reach test.

FIG. 20 is a diagrammatic view of tracking the stretching distance for functional reach test.

FIG. 21 is a diagrammatic view of a training mode to reach to the red area to improve the balance.

FIG. 22 is a diagrammatic view of the results for CTSIB test with component wise break down for sensory inputs.

FIG. 23 is a diagrammatic view of sway plots for CTSIB test.

FIG. 24 is a diagrammatic view of results from functional reach and 4 stage balance.

FIG. 25 is a diagrammatic view of ROME for Home Results being displayed on an external device.

FIG. 26 is a diagrammatic view of ROME for Home individual ADL scores.

FIG. 27 is a flow chart diagrammatic view of ROME.

FIG. 28 is a flow chart diagrammatic view of an Overview of a Point cloud generation and display.

FIG. 29 is a flow chart diagrammatic view of a Process of Unprojection.

FIG. 30 is a flow chart diagrammatic view of a Process of Plane Fitting.

FIG. 31 is a flow chart diagrammatic view of a Process of Rectangle Fitting.

FIG. 32 is a flow chart diagrammatic view of a Process of Sensor Calibration.

FIG. 33 is a flow chart diagrammatic view of a Process to Generate sensor matrix.

FIG. 34 is a flow chart diagrammatic view of a Process of Hill-Climbing.

FIG. 35 is a flow chart diagrammatic view of a Process to Calculate plane-fit error.

FIG. 36 is a flow chart diagrammatic view of a Process to Generate plane matrix.

FIG. 37 is a flow chart diagrammatic view of a Process to Calculate rectangle-fit error.

FIG. 38 is a flow chart diagrammatic view of a Process to Calculate sensor calibration error.

FIG. 39 is a flow chart diagrammatic view of a Process to Transform and project points into 2D (using a sensor's position/orientation).

FIG. 40 is a flow chart diagrammatic view of PT-ROME.

FIG. 41 is a flow chart diagrammatic view of continuing flow chart “1”, as shown in FIG. 40.

FIG. 42 is a flow chart diagrammatic view of continuing flow chart “2”, as shown in FIG. 41.

FIG. 43 is a flow chart diagrammatic view of ROME for Industrial Rehab (Ergonomic Task Training).

FIG. 44 is a flow chart diagrammatic view of continuing flow chart “3”, as shown in FIG. 43.

FIG. 45 is a flow chart diagrammatic view of a Task Efficiency Measurement.

FIG. 46 is a flow chart diagrammatic view of continuing flow charts “4” and “5”, as shown in FIG. 45.

FIG. 47 is a flow chart diagrammatic view of an Assembly Manufacturing Work Flow Analysis.

FIG. 48 is a flow chart diagrammatic view of ROME for Balance.

FIG. 49 is a flow chart diagrammatic view of continuing flow chart “6”, as shown in FIG. 48.

FIG. 50 is a flow chart diagrammatic view of continuing flow chart “7”, as shown in FIG. 48.

FIG. 51 is a flow chart diagrammatic view of continuing flow chart “8”, as shown in FIG. 48.

FIG. 52 is a flow chart diagrammatic view of continuing flow chart “9”, as shown in FIG. 48.

FIG. 53 is a flow chart diagrammatic view of continuing flow chart “10”, as shown in FIG. 48.

FIG. 54 is a flow chart diagrammatic view of continuing flow chart “12”, as shown in FIG. 48.

FIG. 55 is a flow chart diagrammatic view of ROME for ADL and Falls.

### DETAILED DESCRIPTION

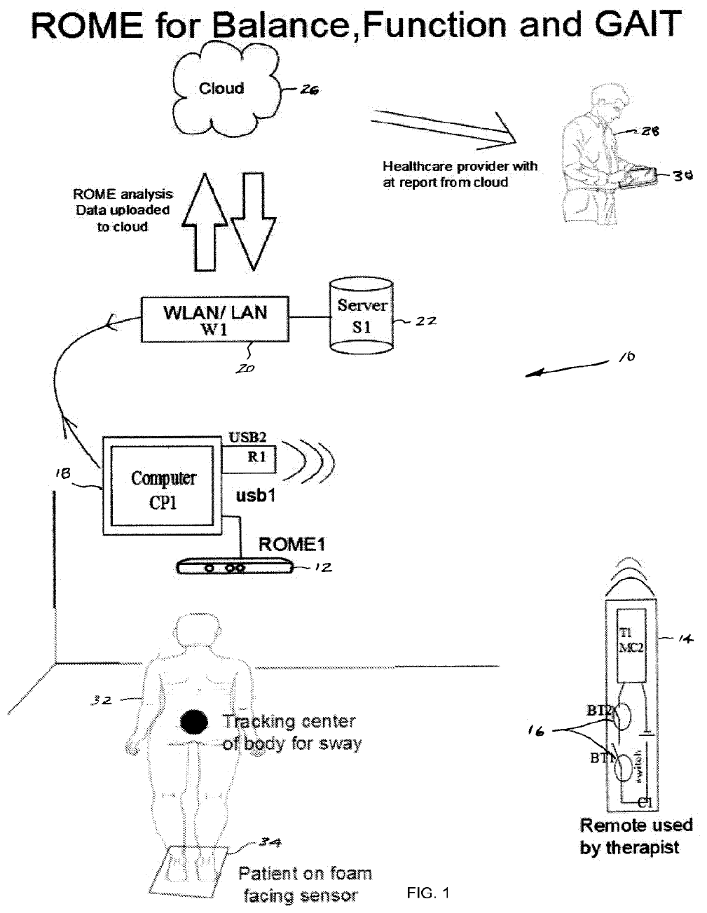

A Balance, Function and GAIT ROME system 10 is shown in FIG. 1. The ROME system 10 comprises a tracking sensor 12, a remote indicator device 14 (e.g. used by therapist) comprising one or more buttons 16, and a computer 18. The tracking sensor 12 is connected to the USB1 port of the computer 18, and a wireless remote device 20 is connected to the USB2 port of the computer 18.

In addition, the system 10 can comprise a network 22 (e.g. WLAN/LAN) and a server 24. The network 20 communicates through the Cloud 26, for example, to a healthcare provider 28 using, for example, a wireless pad device 30 (e.g. I-Pad). The patient or individual 32 stands on a floor sensor 34.

The tracking sensor 12 comprises a projector and a camera. For example, the tracking sensor 12 is a Microsoft-Kinect for Windows, Model L6M-00001.

The remote indicator device 14 wirelessly communicates with the wireless remote sensor device 20. The remote indicator device 14, for example, is a Powerpoint remote device.

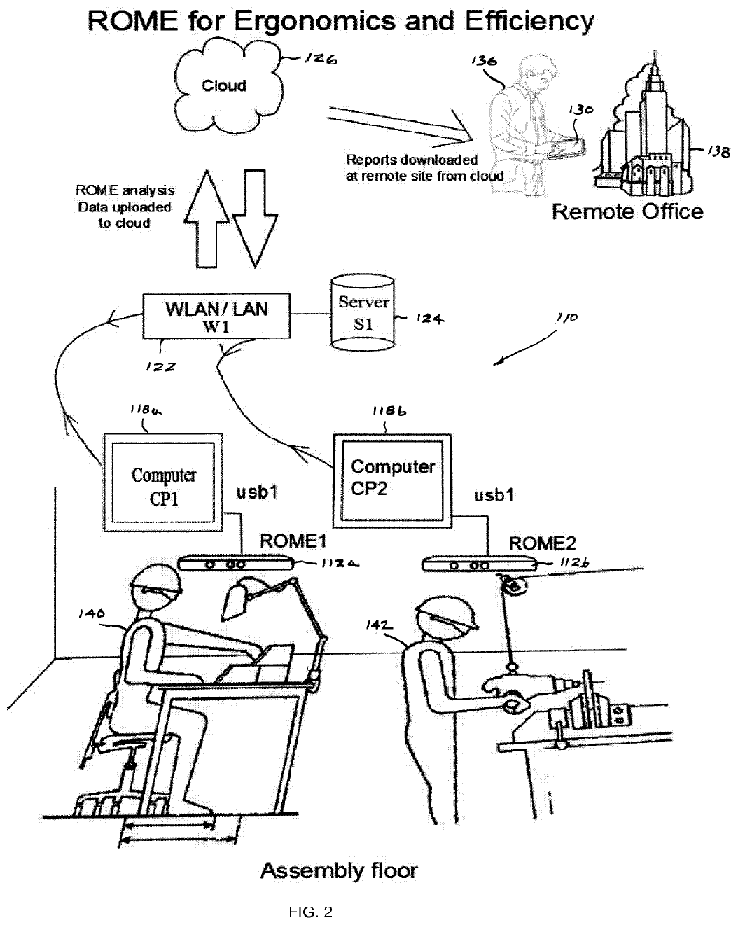

An Ergonomics and Efficiency ROME system 110 is shown in FIG. 2 The ROME system 110 comprises a first tracking sensor 112a, a second tracking sensor 112b, a first computer 118a, and a second computer 118b. The first tracking sensor 112a is connected to the USB1 port of the first computer 118a, and the second tracking sensor 112b is connected to the USB1 port of the second computer 118b.

In addition, the system 110 can comprise a network 122 (e.g. WLAN/LAN) and a server 124. The network 122 communicates through the Cloud 126, for example, to an individual 136 (e.g. evaluator) remote location 138 (e.g. office) using, for example, a wireless pad device 130 (e.g. I-Pad). Workers 38 and 40 are evaluated and/or trained on the assembly floor.

The first tracking sensor 112a and second tracking sensor 112b each comprise a projector and a camera. For example, the tracking sensors 112a and 112b are a Microsoft-Kinect for Windows, Model L6M-00001.

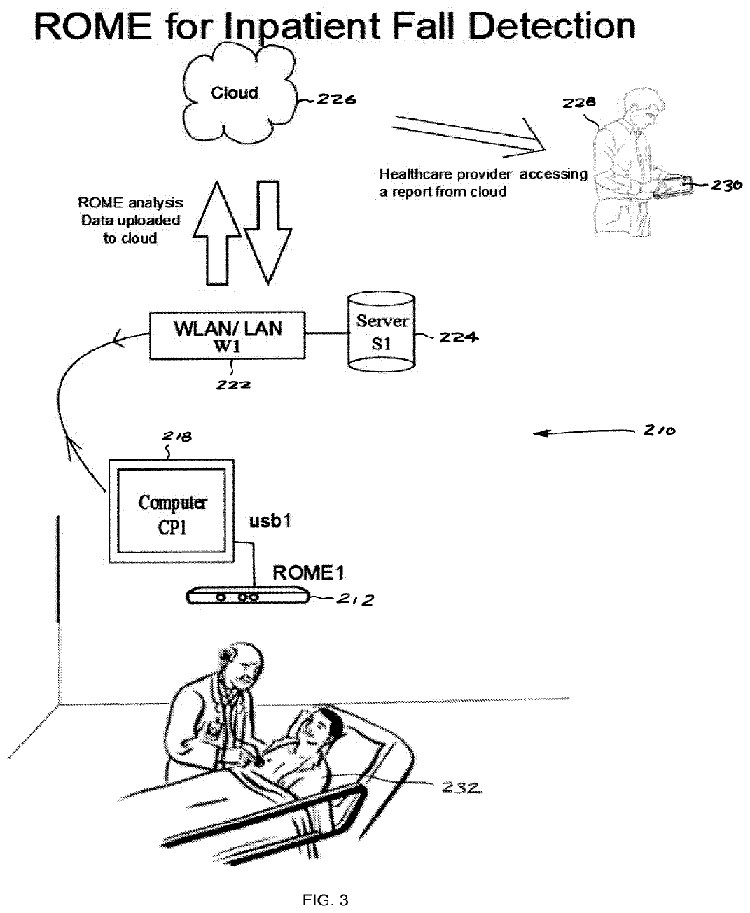

An Inpatient Fall Detection ROME system 210 is shown in FIG. 3 The ROME system 210 comprises a tracking sensor 212, a second tracking sensor 212b, and a computer 218. The tracking sensor 212 is connected to the USB1 port of the computer 218.

In addition, the system 210 can comprise a network 222 (e.g. WLAN/LAN) and a server 224. The network 222 communicates through the Cloud 226, for example, to a healthcare provider 228, for example, a wireless pad device 230 (e.g. I-Pad). The patient 232 is remotely monitored in this manner.

The tracking sensor 212 comprises a projector and a camera. For example, the tracking sensor 212 is a Microsoft-Kinect for Windows, Model L6M-00001.

ROME captures the actions performed by a person using a markerless tracking sensor. This data can be used for ergonomics, physical therapy or GAIT analysis.

In ergonomics, this system can be used to provide information on how a person is performing an action, which can be used to monitor and automatically train them in how to perform a manufacturing job ergonomically.

In physical therapy, this system can be combined with a pain indicator device to perform Range of Motion testing with automatic pain recording. For GAIT analysis, this system can monitor a person's motion in real time and log this for review.

The purpose of ROME is to capture human interaction and motion in three (3) dimensions with applications in industrial, medical simulation and rehabilitation. The system tracks landmarks on multiple people in real time, based on IR depth mapping technology. The multiple applications for ROME including: ergonomics and safety assessment, ergonomic task training, task efficiency measurement, assembly or manufacturing work flow analysis, telemetric ergonomic assessment and training, physical therapy and GAIT Analysis.

### Sensor Calibration

Calibrating the ROME sensor provides capabilities to segment regions based on height and depth in real world coordinates. When using multiple ROME sensors to cover a wider area or for tracking the same person/object across multiple depth images, calibration needs to be performed to integrate the depth sensors data into real-world coordinates.

### Calibration of a Single ROME Sensor:

In order to calibrate a ROME sensor, a large, flat, rectangular object is placed in view of the sensor, and one corner of the object is designated as the origin of the world coordinate space. A single depth frame from the ROME sensor is captured and saved to a file. This file is read by an interactive calibration program, “depth-view”, to perform the calibration. The user marks an area of the depth image that belongs to the calibration rectangle, and presses a button for depth-view to fit a plane to it. The user then has the plane expanded to cover the entire rectangle. The user then has depth-view fit a rectangle to the previously expanded plane. The user then selects the origin corner of the rectangle and has depth-view to export the rectangle's coordinates. The user loads each of these coordinate files, one at a time, into another program called “ir-calibration”. When a file is loaded, the user gives an estimate of the sensor's location, and asks ir-calibration to calibrate the sensor. If the calibration is satisfactory, the user has ir-calibration export it to a file, which can be loaded by ROME multi-server for use at runtime.

### Plane Fitting:

To fit a plane to a set of pixels in the depth image, the pixels are first unprojected into 3D points based on the field of view of the ROME sensor, which generates a 3D point cloud in sensor space. A 2-dimensional hill-climbing algorithm is then used to find a normal for the plane passing through the centroid of these points.

To fit a plane to these points, the identified points are first averaged to obtain the position of the centroid. It is assumed that the fitted plane will pass through this centroid. To start the hill-climbing algorithm, a normal is assumed that starts with a horizontal plane passing through the centroid of the points. An error value is then calculated by summing the distance each point is away from the estimated plane. The program attempts to minimize this error value by iteratively adjusting the plane in each dimension. The program starts with a movement factor T of 1.0. The program then adjusts the plane's normal in the X and Z dimensions and recalculates the error. If the error is smaller, we accept the new plane and continue adjusting. Once a local minimum error has been found, f is divided by 2, and the plane is continued to be adjusted, until f is less than or equal to epsilon. For this application, epsilon is chosen to be zero.

### Plane Expansion:

Once the plane has been fit to a small area of the depth image, an iterative flood-filling algorithm is used to expand the plane to the rest of the calibration rectangle.

This algorithm operates on a queue of X-Y pairs. First, all the selected pixels from the previous plane-fitting step are pushed into the queue. Next, an array is initialized to mark whether a pixel in the depth image has been selected as being part of the calibration object.

While pixels are in the queue, a pixel is taken from the queue and its point cloud distance is tested to the closest point on the identified plane. If this distance is within a given threshold and the point is not already marked, the point is marked in the array of selected pixels. The point is also added to the set of selected points, and the neighboring points, one in each of the 4 cardinal directions, are pushed onto the queue.

After the plane has been expanded to cover the entire calibration rectangle, a new plane is fitted to the newly selected points. This should improve the calibration precision slightly.

### Rectangle Fitting:

Once there is a plane that covers the entire calibration rectangle, the software will fit a rectangle to the calibration rectangle. A 1-dimensional hill-climbing algorithm is used to rotate a bounding box around the plane's points until the box has a minimum area.

### Sensor Calibration:

Once the rectangle is fit to the depth image, and the user has designated one corner as the origin of world space, the sensor's position and orientation are calibrated. The user inputs an estimate of the sensor's location and may interactively adjust the orientation to make sure the calibration points have been loaded properly. The calibration algorithm then uses a 6-dimensional hill-climbing algorithm to minimize the error between the image shown on a virtual sensor that operates in world space, and the rectangle's coordinates projected into 2D.

### Multiple ROME Units and Point Cloud Integration:

Each computer running ROME skeleton-record sends a depth frame over the network. The server computer running ROME multi-server or a similar program unprojects these frames into a 3D point clouds, then transforms each point cloud based on the calibration of its sensor. Each point cloud is drawn individually using OpenGL point sprites. Currently, all sensors must be calibrated using the same calibration surface in the same position. This allows to convert data from any sensor's coordinate space into a unified world space.

In short, the data from the sensor is in the sensor's coordinate space and if the sensor is tilted or rotated, the data will appear rotated in the opposite direction. Also, the origin of the data, the coordinate (0, 0, 0), is always at the sensor's position, so data from several sensors is not aligned. The calibration process calculates a transform for each sensor relative to a real-world calibration object such as a table. In addition, when the inverse of this transform is applied to the image data, the data is rotated and translated “backwards” from the sensor's space into world space. The calibration process also presents the data in world space, with the origin (0, 0, 0) located on a corner of the calibration object. This makes the display more natural, because world-space data will not be tilted or rotated even if the sensor is. This also allows the data from multiple sensors to be aligned and shown on the same display.

### Rome—Industrial Rehabilitation and Ergonomics

### Ergonomics and Safety Assessment:

ROME can be used to capture the actions and movements of a person while performing a task. This allows a system to be designed to improve the ergonomics and safety of performing manufacturing and assembly jobs. ROME can record a person performing a task properly, and allow an ergonomics person to create a template file of the correct actions that should be performed when doing a task, which is dubbed an “ergo template” for a task. After the ergo templates are generated, workers can be brought in to be evaluated by the ROME system. The workers' actions will be compared with the correct ergonomic process identified in the ergo template for someone fitting the workers build and medical conditions, and any deviations or differences can be identified. This information would then be provided to the management or ergonomics personnel to determine if training or corrective actions are necessary to improve the health and decrease long term ergonomic impact on the individual.

### Ergonomic Task Training:

Extending the Ergonomics and Safety Assessment system, a task training system can be developed to train workers to perform actions in the ergonomically correct fashion. This can be used to train new workers, maintain proper ergonomics of workers over extended periods of time, and assist workers returning from injuries on any changes in action they should perform. An example of this application consists of a worker being brought to a small mock-up assembly area for a task they are to be trained on. In this area, a display system can show an animation of an avatar performing the task, and alert the worker if they are performing the actions incorrectly or in a non-ergonomic fashion. This setup can be created for each task a worker would perform, and monitor and record all the training sessions for the training history of the worker. Ergonomics personnel could also review each training session and provide more detailed training, if necessary.

### Task Efficiency Measurement:

Another application of the ergonomics and safety assessment would be to capture information about how well a person can perform a task. This information is currently evaluated by industrial rehabilitation centers to show that a worker is eligible to start or return to work after an injury. ROME can capture this information automatically while a worker performs a task, and assess the performance for metrics like speed, accuracy and quality. This data can be used in multiple ways, from assessing if the worker is fit to be employed and providing necessary information for insurance companies, to determining if workers are fit to return to work after being injured. This system could also be set up on an active assembly line to monitor performance of workers over a period of weeks and months to provide objective evidence that the worker may or may not be injured.

### Assembly/Manufacturing Work Flow Analysis:

As ROME can track multiple people simultaneously, the system can also be used to assess individual and group performance during specific tasks. This information can be used to train workers better, discover improved techniques that can be used elsewhere, and to show how effective each individual is at in a group assembly process. Using multiple ROME units, this analysis can be extended throughout an entire assembly line to provide metrics to management about the efficiency of the production line, automatically. If work flow was slowing down in a specific part of the line, corrective actions could be taken earlier to improve the situation before any major production disruption occurs.

### Telemetric Ergonomic Assessment and Training:

The ROME industrial rehabilitation and ergonomics system transmit information over a network to provide near real-time information about remote manufacturing sites to corporate ergonomists, reducing expensive travel costs incurred from personal visits. This would allow equivalent safety and ergonomic training opportunities to all manufacturers at a company, improve health and safety of individuals, and teach each worker the appropriate skills needed, regardless of where they are located.

### Additional Applications of ROME for Industrial Rehabilitation and Ergonomics Include the Following:

ROME captured data can be used to perform ergonomic and safety analysis. While performing a task, ROME can automatically record and store movement of various joints while a job task is accomplished in an ergonomic and efficient way. This data will be reviewed by an ergonomist to create an ergo-template.

ROME can be used to compare workers actions with the ergo-template to generate deviation metrics while a worker performs a job. This data can be analyzed by an ergonomist, safety engineer, or an industrial rehabilitation therapist to determine the points of impact on a joint and design an alternative way to perform the same task with a less strenuous approach.

ROME can be used to design adaptive job training programs to self train and evaluate employees to ensure they are performing their tasks in an ergonomic, safe and/or productive fashion. During the training and evaluation process, ROME will automatically measure metrics real-time and provide constructive feedback to improve the safety of the worker.

ROME can automatically screen and evaluate the efficiency of workers before hiring them for certain manufacturing floor tasks, evaluate the efficiency of a worker after returning back to work from an injury, and/or evaluate the efficiency of the worker on the factory floor.

ROME can work in a remote configuration, where the device is hooked to a computer with a high-speed internet connection to transmit information from the patient to the physical therapist.

### PT-ROME

When a patient comes to physical therapy (PT), one of the standard tests performed is a Range Of Motion (ROM) to determine what is injured and to track the progress of treatment. The physical therapist or assistant uses a tool called a goniometer to measure a single angle and observe for any abnormalities during the performance of the motion. As the ROME system tracks multiple points simultaneously, these static angles can be captured along with information based on fluidity and rapidness of motion. The ROME for a physical therapy package also adds a patient pain indicator device, to capture at what points the patient encountered pain during an action and the intensity of the pain on the NRPS scale. The PT-ROME package stores this data and calculates a Pain-Motion Index (PMI) to show the treatment progression over time. This system provides the ability to capture the position, angle and fluidity of action of multiple joints during ROM evaluation, as well as the capturing of the patient's pain level during the procedure.

### PT-ROME for Gait

When a GAIT analysis is needed, PT-ROME for GAIT would be used. The patient may be suffering from Alzheimer's disease, which would affect their ability to live independently. The patient may have had a concussion and are unable to maintain their balance, is being fitted for a custom prosthetic or orthotic after loss of a limb, or may be undergoing assessment to determine treatment for joint problems at the hip, knee or ankle. During the PT-ROME for GAIT exam, the patient would stand, walk or perform other actions, like standing up from a sitting position in a chair, and the 3-dimensional data would be recorded for these actions. While performing the actions, if the patient experiences pain they have a pain indicator device to press to record the time of pain to show which action caused pain. After performing the PT-ROME-GAIT exam, the video of the patient's skeleton would be stored and available for review. Base line data can be established in the initial visit and the progress in the functional condition of the patient can be objectified.

### Timed Up and go (TUG)

When a patient performs a TUG test, ROME for balance automatically tracks the movement of all the limbs on the patient, measures sway of the center of the body before and while getting up from the chair, measures the sway, velocity and acceleration of the patient. The system also tracks the spine, hand movement, upper-lower body coordination and head position of the patient in 3D to determine their strategy while getting up from the chair. Rome for balance also tracks the sway of the patient after they leave the chair support.

When a patient is walking in the TUG test, ROME estimates number of steps, stride length, height of each step, symmetry of placing legs, pain level of the patient, leg movement while turning 180 degrees, sway, total time taken, walking stance and step continuity real-time. These metrics are ranked against a population and are compared against norms for that selected range of age, conditions and treatment applications.

Measuring Balance with Modified CTSIB and Functional Tests:

Balance is a person's ability to maintain or restore equilibrium state of upright stance, without having to change the base of support. The central nervous system monitors the status of body and external environment through three mechanisms of peripheral sensation. ROME quantifies a patient's ability to maintain upright posture based on the sensory inputs from visual, somatosensory and vestibular systems. The system evaluates a patient based on four different test conditions that last for a customizable time frame, in this case 20 seconds each while the patient's legs are next to each other. In condition one, the patient stands still on the floor with his eyes open, using all three sensory inputs. In condition two the patient stands still on the floor with his eyes closed, using somatosensory and vestibular systems. In condition three, the patient stands still on dense foam with eyes open, using vestibular and visual senses to maintain balance. In last condition, the patient stands still on dense foam, with eyes closed, using the vestibular sense to maintain balance.

The sway of a patient from top view is depicted in a radial plot in FIG. 2) for the all the four conditions. The concentric circles help therapists identify the range of the sway of the patient. The ROME sensor detects and measures key parameters that include cumulative sway, sway velocity, range of sway in multiple planes of motion, upper and lower body motion, and time spent without losing balance to generate a graded scale for each of the sensory responses and the overall score of the patient. The patient response is graded on a scale from 0, a condition representing fall, to 100, representing no sway during the 20 second period of each test. The posture moment in the four conditions will determine the particular patient's strategy in using one or more of their sensory systems to stay in balance. A typical result sheet would look like FIG. 3). Weaknesses in vestibular balance can be objectively measured. These tests, when performed in a baseline scenario and over the course of treatment, can help gauge the effectiveness of the treatment plan.

### Functional Reach Test:

The functional reach test is a clinical measure intended to assess dynamic balance. This test measures the maximum distance a patient can reach forward beyond the arm's length while maintaining both feet on the ground in a standing position. Typically, yard sticks mounted on the wall at shoulder height are used to measure the total reach. ROME automatically measures the patient's ability to reach the maximum distance from their arms length and also measures the time it takes to reach that distance. ROME automatically adjusts for limitations in shoulder flexion to record an accurate measure of forward excursion.

### Monitoring Activities of Daily Living (ADL):

ROME is used to monitor the Activities of Daily Living. ROME sensors are placed in multiple settings in the house, hospital or in a nursing home setting. The sensors are typically positioned to capture the living room space, kitchen, bedroom and the rest room. The sensors record the movement of multiple people separately and automatically keep track of their body position, location in the room and interaction with other people. This data is stored on the local computer and uploaded to a cloud and saved in a highly secure 128 bit encrypted server. ROME, with its pattern recognition capability, can identify when a fall happens or when there is a reduced activity without the patient pressing a button. When a fall is detected, an emergency alarm signal is sent to a monitoring agent, who can then communicate directly with the patient through the microphones and speaker on the sensors. ROME for Home will monitor patients continuously throughout the day. Examples of activities that ROME can capture, analyze, and generate report include, but are not limited to, bathing, dressing, transferring, using the toilet, continence, and eating. Based on their activity, ROME will create a probabilistic model that can identify patients with increased fall risk before a fall happens based on a particular deviation level from accepted probabilistic range. ROME can transfer information regarding the reduced physical activities in a timely fashion to help the clinician in charge to reach a treatment decision. ROME can transfer information regarding the reduced physical activities in a timely fashion to help the clinician in charge to reach a treatment decision.

### Additional Description of ROME for Physical Therapy:

ROME allows a physical therapist or assistant to pre-select the routine and monitor the results through a live streaming video or through a recorded file to make diagnosis or to follow up on status of patients undergoing treatment.

The current standard of diagnosis to evaluate the state of joint problems includes the use of goniometer to provide the maximum angle the patient can flex, bend, extend or rotate with respect to a joint. ROME provides a continuous profile of motion of multiple joint movements used to calculate joint angle up to 30 Hz. The therapist need not be engaged to calculate the angle measurements. The patient can press a wireless button once or multiple times to indicate the pain while performing a particular maneuver. The angles at which the patient encountered pain are stored and the patient will identify the pain level on a NRPS scale. The number of pain points, the corresponding pain level and the normal range of the overall joint movement will be used to calculate the Pain Motion Index (PMI) per joint.

The combination of the PMI values for a particular joint over time shows the effect and progress accomplished through the treatment plan.

The PMI over multiple visits can be used to alter treatment plans if the current plan is not effective. The PMI for each joint ROM over multiple visits can be captured in a report, which includes a combination of graphical representations, tabulated data of the patient's progress, and a stick figure depiction of pain. The PMI report can be attached to the treatment notes of the therapist, which can be used by a primary care physician or an orthopedic specialist or to the insurance provider to evaluate the patient's progress.

Physical therapists normally document their findings with single angle measurements for joint motion, as well as the patient's response saying it hurts at the end of the entire procedure, which only tells part of the story. By using the ROME software during an evaluation, the therapist can better document issues in an effective, automated and streamlined fashion. The objectively recorded data about patient evaluation can be used to discourage fraud and malpractice, as well as providing better records for insurance companies' audits. Fraudulent workman's comp cases can be identified by inconsistent PMI readings over the course of the treatment.

In addition, ROME can capture objective data for multiple patients over multiple visits based on numerous conditions to prove or disprove the effectiveness of a treatment thereby facilitating result-driven healthcare.

A new trend in the insurance world is to move towards result driven healthcare instead of repeatedly paying for ineffective treatments. ROME can capture objective data for multiple patients over multiple visits based on numerous conditions to prove or disprove the effectiveness of a treatment.

### Analytics for Rome

Data from ROME can aid in the evaluation of workers compensation claims by quantifying the intensities of pain experienced while performing certain actions over multiple sessions. This data, when combined with physical therapists interpretation, can be used to figure out the validity of a claim. It would be hard to fake pain at the same angle of an action over multiple visits. Inconsistent readings during and between visits could indicate a fraudulent claim of injury.

The data collected from multiple therapy centers by multiple therapists on different patient groups can be subcategorized and analyzed to measure efficacy of a therapist, efficacy of an institution, or efficacy of particular treatment plan based on age, sex and problem. This could potentially help to streamline the treatment plans across multiple organizations.

Data from ROME industrial rehabilitation can also be used to determine the root cause for injuries while working on a specific job tasks. Using this, corrective training can be determined to improve safety of workers.

### Metrics

Using prior art algorithms, the ROME sensor outputs estimated positions of a person's skeletal joints, such as hands, elbows, shoulders, feet, knees, hips, and head. These positions are in 3-dimensions. Given a start and end position, the displacement of a joint is calculated by performing vector subtraction. The Euclidean distance is calculated directly from the displacement. The average speed of the joint is calculated by dividing distance by time. An instantaneous linear velocity for a joint for a single frame is estimated using numerical differentiation of the joint's position with respect to time. Acceleration of a joint is estimated by calculating the second derivative of joint position. Given 2 joints and a plane, such as M2, M0, and the XY or YZ plane, an angle is calculated from the horizontal or the vertical. Subtracting M0 from M2 in the plane, creates a 2D vector along the person's spine. The angle of this vector is calculated using the a tan 2 arctangent function. Given 3 joints A, B, and C, we calculate the angle ABC by calculating D=B−A, and E=C−B, taking the dot product of normalized D and normalized E, and calculating the arccosine. This angle will be independent of the axes, and is used easily for arms or legs. Similarly to linear velocity, angular velocity is estimated by numerical differentiation of an angle.

### Components & Use

### ROME—Industrial Rehabilitation and Ergonomics:

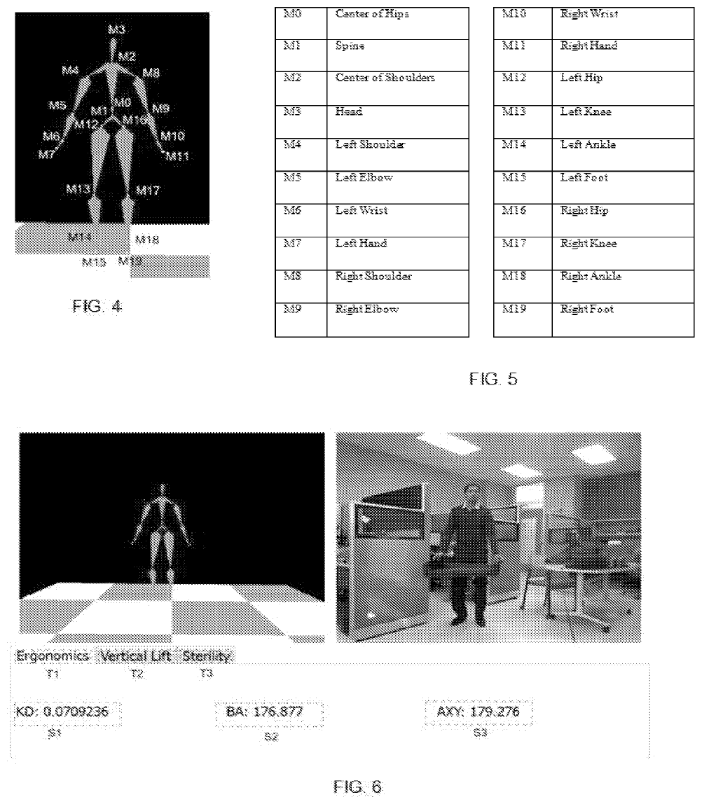

Applications of ROME in industrial rehabilitation and ergonomics are based on the sensor that tracks a person's interaction with the environment. Information is provided in the form of 3-dimensional points of a person. Again, the ROME for Ergonomics and Efficiency is shown in FIG. 2. The tracked points of the person are shown in FIG. 4, and the identification of the tracked points is shown in FIG. 5.

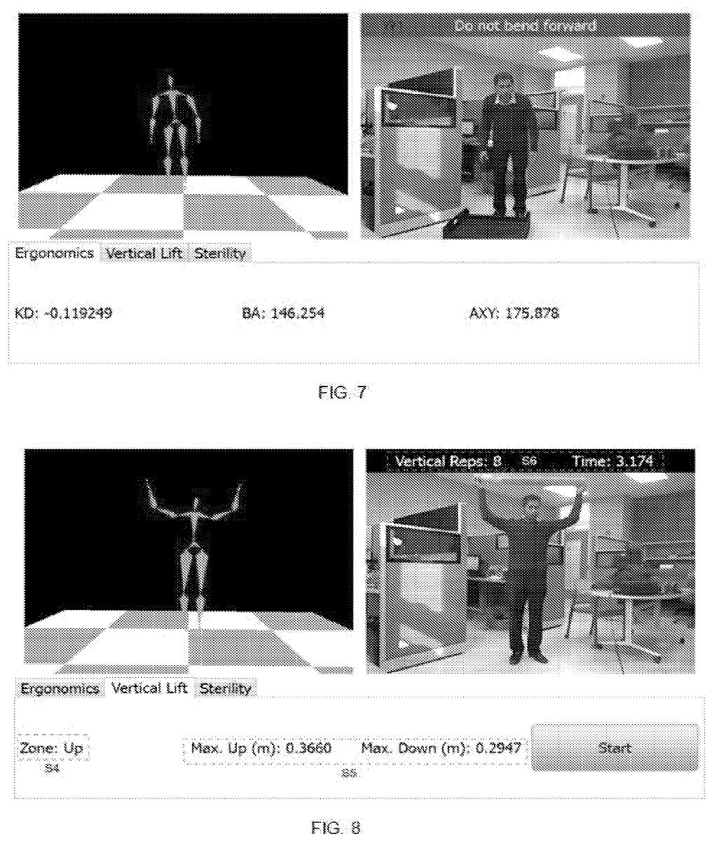

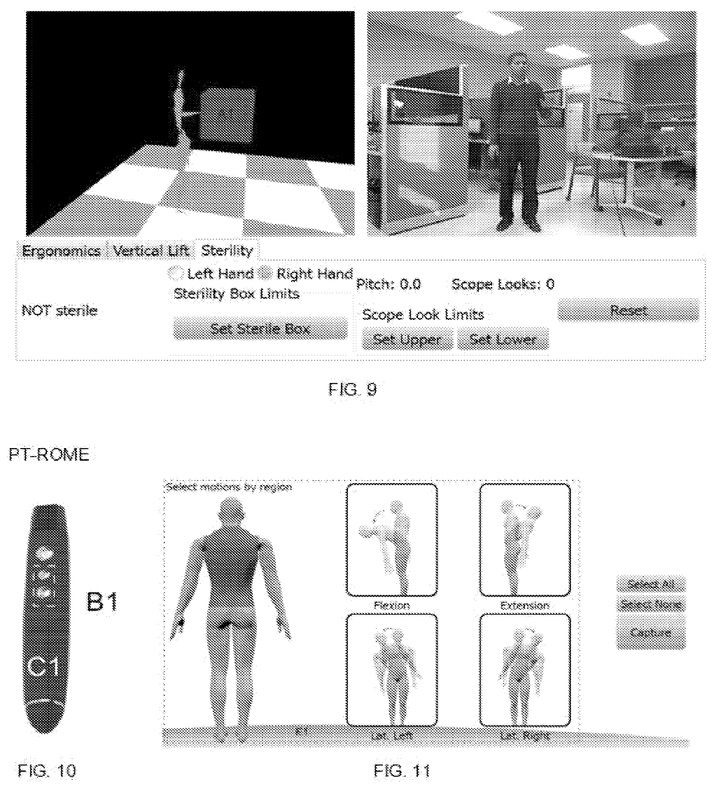

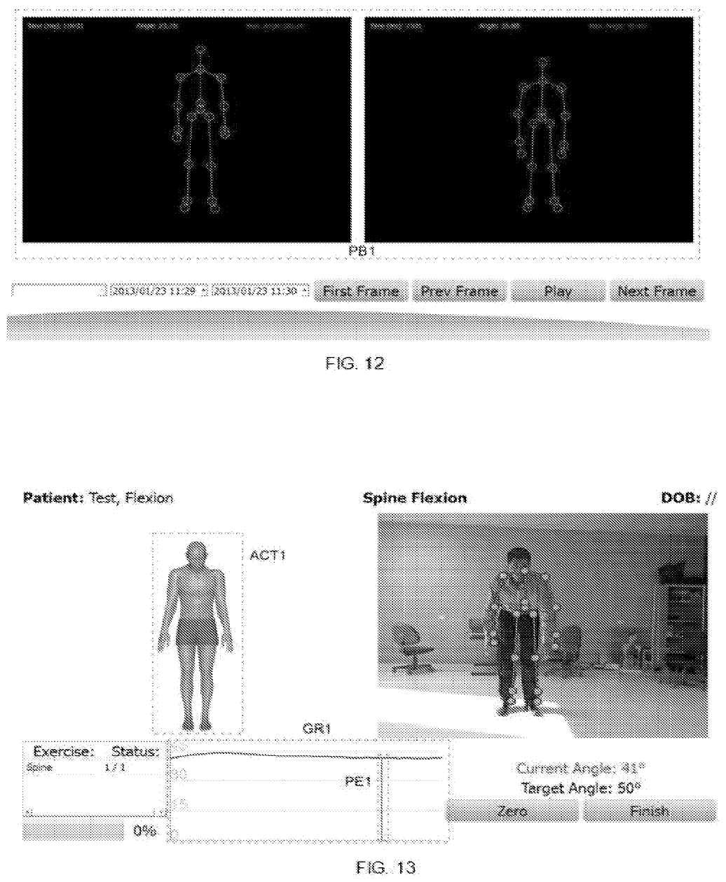

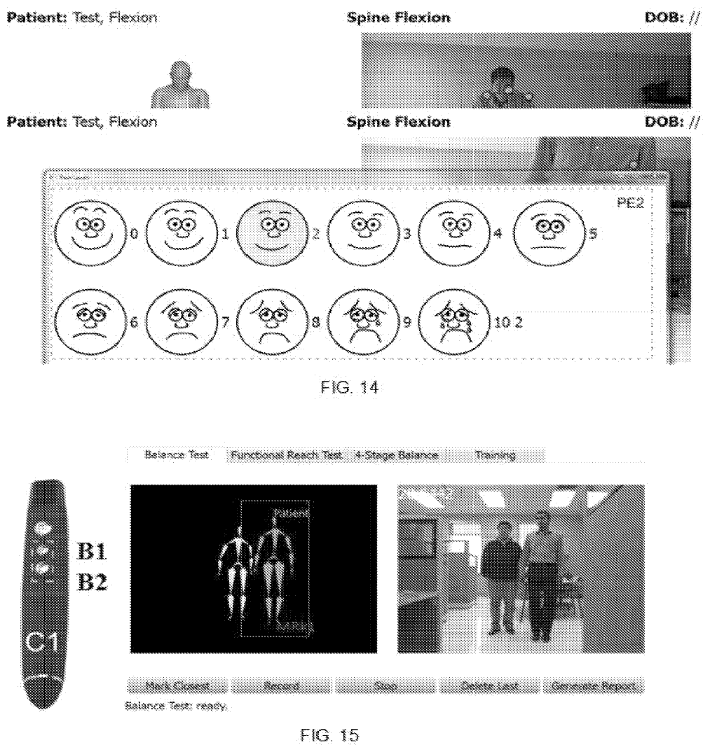

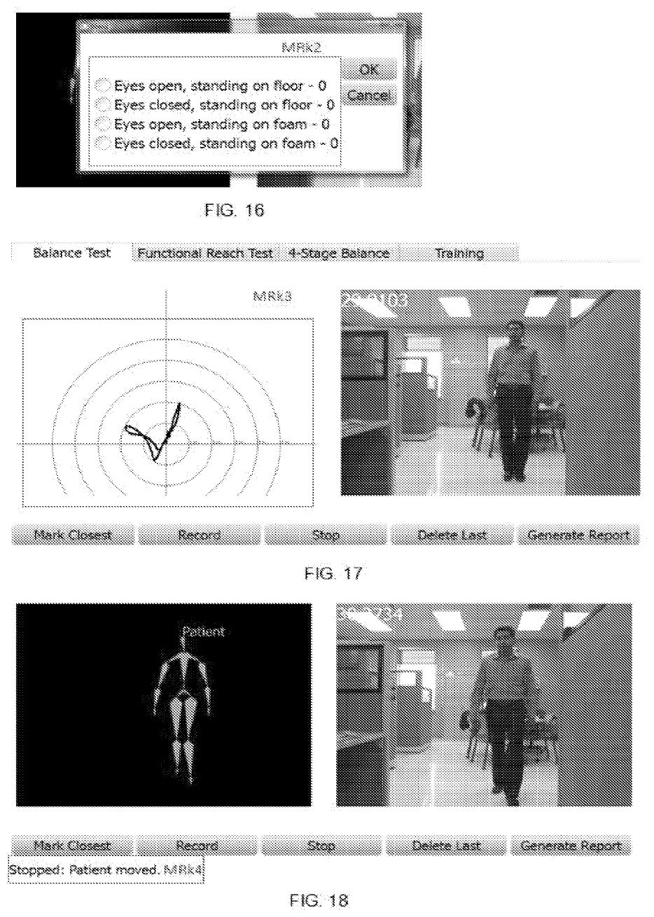

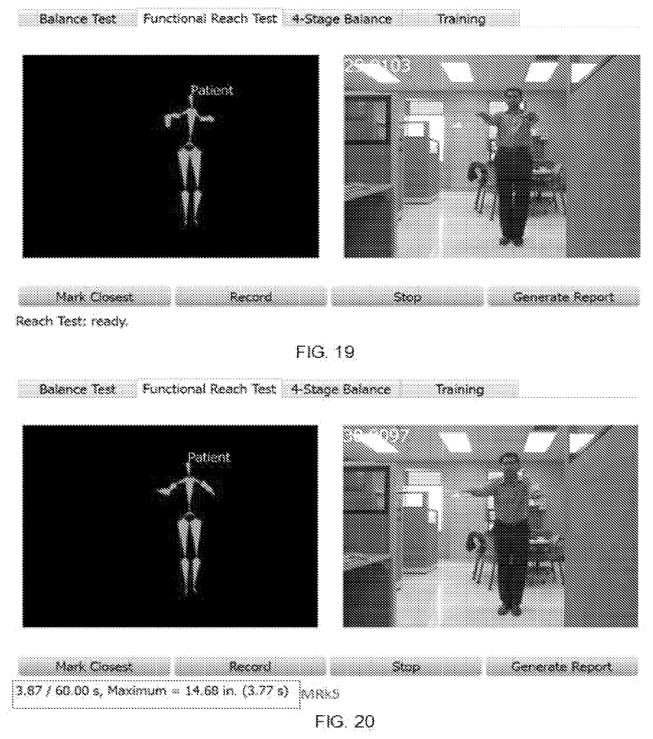

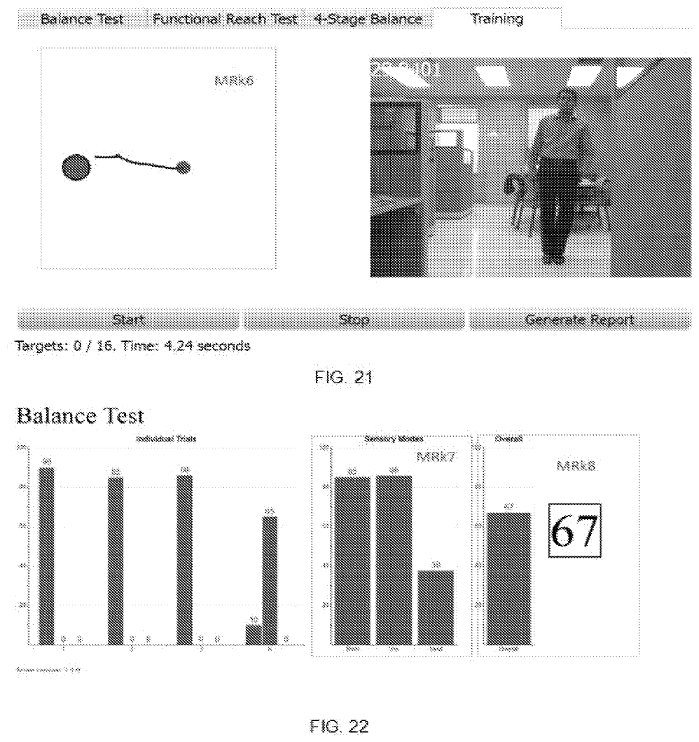

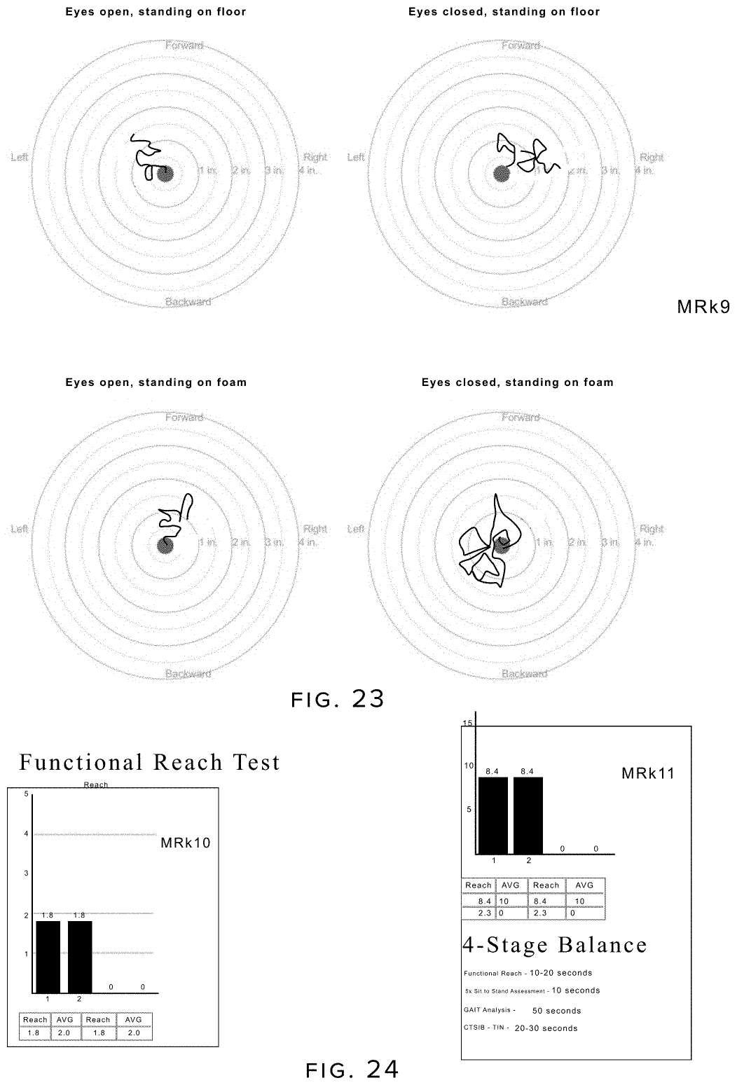

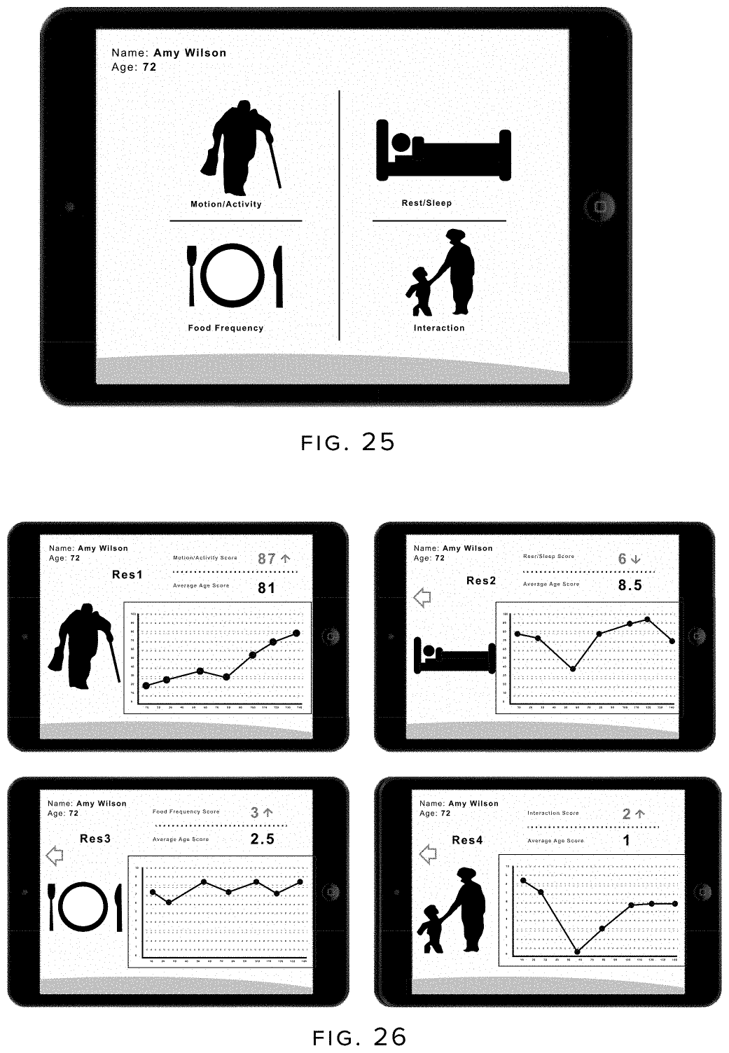

Using this information, three (3) programs have been developed, including an ergonomic task trainer (FIG. 7), a task efficiency measurement program (FIG. 8), and a sterile area notification system (FIG. 9).

The ROME system 110 comprises the sensors 112a, 112b that track multiple people's skeletons, and provides spatial coordinates of certain regions of their body. Information for individual skeletons is passed as a group of points corresponding to certain elements of a person (M0-M19), as shown in FIG. 5. This data can be processed by three (3) software programs: an ergonomic task trainer (T1), a task efficiency measuring program (T2), and an area sterility program (T3), as shown in FIG. 6. In the ergonomic task trainer, metrics are calculated and displayed in real time about how well a person is performing a task.

For the ergonomic task trainer (T1), the system calculates metrics for how much the knee is bending (S1), how the back is bending (S2), and how far to the sides the person is bending (S3). If these metrics are too far out of bounds for the ergonomic way of performing a task, a warning message is displayed on screen (W1). For the task efficiency measuring program (T2), the positioning of the persons hands (S4), minimum and maximum movement of hands (S5) and number of repetitions in a fixed time period (S6) are shown. For the area sterility program, a clean area (A1) is designated and a warning tone is played every time the person enters the area.

The task trainer (T1) provides feedback to a person when they perform a task in a non-ergonomic fashion. The person operates in the visible space of the sensor, and based on the information provided about the skeleton, their actions can be monitored. The task this system currently performs is verifying that employees can bend over and pick a large or heavy object up off of the ground without placing excessive stress on their spine. Using points M0, M1 and M2, an angle for spine bend is calculated (S2 and S3), and using information about M12, M13, M14, M16, M17 and M18 the bend of the knees can be tracked (S1). As the person performs a task, if these metrics deviate substantially from the accepted practice (i.e. do not bend forward more than 20 degrees), a warning message (W1) is shown to indicate that the person is doing something improperly. Through repeated training, the person should be able to stay within the acceptable limits, therefore improving how ergonomically they are performing the task.

The task-efficiency measuring program (T2) is designed to monitor how fast and accurately a person can perform a repetitive task. This program measures how fast an employee can move a bar from below their waist to above their head, back to below their waist in a finite period of time. If a person has to take something off a high or low shelf repeatedly, they need to actually be able to perform that action rapidly. Using the information from the sensor 12 about M7 and M11, the actions of the person can be derived into metrics.

The metrics are derived from observing the person, such as which zone they are in (S4), minimum and maximum travel of their hands (S5) and number of successful repetitions (S6). With this program, a manufacturer would have a specified minimum number of actions that must be accomplished in the timeframe. If the person did not meet or exceed the necessary number of actions, they would not be eligible for employment or be qualified to return to work.

The sterile area monitoring program (T3) is designed to notify any person when they breech an area that has been designated as a sterile area. For this program, an area is indicated and set to be sterile (A1), and any time a tracked person enters the area, a warning sound is played. The goal of this program is to raise awareness of people that they are interacting in a region and possibly affecting tools or sterility of devices contained therein.

For industrial rehabilitation and ergonomics, ROME allows for objective data capture for measuring interactions with machines in assembly line, warehouse and manufacturing settings with marker-less sensors that do not interfere with the workers actions. ROME allows for creation of a template or multiple templates that indicate an ergonomic, safe and efficient way of performing a job and the automatic derivation of metrics from the templates to create rule sets. ROME also allows for evaluating multiple workers with respect to the templates and rule sets, using classifiers and generating an automatic/semi-automatic way to ergonomic metrics, as well as generating concise evaluation reports that can be provided to improve inter-department communication and workers safety.

In addition, ROME provides an avatar based self-training module to train the workers in an adaptive fashion on improved ways of performing the job and allows for using collected data to document evidence of evaluations performed on worker and document improvements attained through self-training process for possible uses relating to workers compensation cases or insurance fraud. These can be performed remotely at multiple locations without the physical presence of an ergonomist. ROME also enables recording joint movement while performing job functions in 3-D and play it back in a 3-D viewer, as well as allowing for visual comparison (side by side) of this movement from multiple time points or across multiple people.

Contrary to existing practices in which ergonomics personnel physically observe the performance of a person, or a video recording system that monitors a person to extract the same metrics, the ROME system allows for automatic data capturing and processing, and can be easily deployed in remote facilities. This decreases significant amounts of travel time, as they allow for automatic collection of metrics the ergonomics personnel would collect manually. This system also captures and records data, which can be useful for employment records or to satisfy regulatory or legal needs. The self-training aspect of the ergonomics task trainer is also more likely to help improve a workers ergonomic health, as the training can be done cheaply and regularly.

### ROME in Physiotherapy

The PT-ROME system comprises the tracking sensor and a patient pain indicator device, as shown in FIG. 10. The patient pain indicator device is a handheld wireless device comprising a button to indicate when pain is felt. The software package connects and interprets information from the sensor and pain indicator, as well as manages the flow of an examination. Once a therapist selects a patient, they can look at a playback of past visits (PB1) to see how treatment is progressing. After the therapist reviews this, they can select what evaluations the patient will perform (E1). During the examination, an avatar shows the bending action that the patient to perform (ACT1). The angle the patient is bending is plotted (GR1) in real time and any pain events (PE1) are shown. After a pain event is triggered, a pain event scale screen (PE2) records the intensity of the pain.

With PT-ROME, a patient is going to perform specific actions to test how far they can move parts of their body, to track treatment progress of physical therapy. PT-ROME will have the therapist log in and enter the patient info, if necessary, and allow the therapist to review past sessions of the patient through a recorded skeleton playback system (PB1). Once the therapist is caught up on the state of the patient, they can select which Range of Motion actions for the patient to perform (E1). The patient is given a pain indicator device 14 and instructed to press the button 16 when they encounter pain when performing the actions. The screen displays a virtual avatar of a person performing the desired action (ACT1), and the calculated bend angle is plotted on screen (GR1) along with any pain events (PE1) from the patient pressing the pain indicator button (B1).

If a patient indicates pain during a test, a pain input screen (PE2) is shown after completing the current examination. This captures how intense the pain of the patient is on a standard pain scale. After completing all of the selected evaluations, a report is generated capturing things like maximum angle reached, intensity of pain and other parameters to aid the physical therapist. With this information, they can make treatment decisions and proceed with the rehabilitation exercises for the patient.

PT-ROME enables objective data capture of joint angles exerted during a evaluation of a range of motion of the patient with unobtrusive marker-less sensors that do not interfere with the therapist's ability to observe. PT-ROME also provides a database with the recorded evaluation history for a patient at each step during treatment that is accessible during treatment and evaluation, and allows for capturing at what time during an examination the patient is experiencing pain using a patient operated device. Using this information, metrics about treatment progress and patient condition, as well as concise evaluation reports that can be provided to physicians and patients to improve communication and patient treatment process can be generated. The collected data also can be used as evidence for whether a patient has a medical condition, including possible uses relating to legal cases or insurance fraud.

Contrary to existing practice in which a physical therapist or assistant uses a goniometer to manually measure each angle and document any abnormalities during performance of an action, the PT-ROME system captures this data automatically, and can provide information about how fluid a motion is and provide a recorded video of patient data for the therapist to review. Combining information about the maximum achieved angle and the intensity of pain experienced by the patient, metrics are derived to help show the progress of treatment for the therapists.

### ROME in GAIT Analysis

PT-ROME-GAIT uses the tracking sensor and patient pain indicator device (FIG. 10) to provide metrics about how a person stands, moves and performs other actions. The pain indicator device is a handheld wireless device comprising the buttons to indicate when pain is felt. The software package connects and interprets information from the sensor and pain indicator, and records videos of how the patient moves and interacts. When a gait analysis needs to be performed, a patient would be placed in front of the PT-ROME-GAIT system and instructed in the actions they should perform. While performing the gait analysis actions (normally standing or running on a treadmill), the software will record the position and movement of the tracked points in 3D. After enough data has been captured, a trained professional can review the videos and determine the course of action to follow. Later in the development, automated algorithms can be designed to calculate and check for basic problems and offer easier assessment for the professional.

The PT-ROME-GAIT system allows for objective data capture of joint angles exerted during the performance of a gait assessment using with unobtrusive marker-less sensors that do not interfere with the therapist's ability to observe. PT-ROME-GAIT provides a database with the recorded evaluation history for a patient at each step during treatment that is accessible during treatment and evaluation. It enables the capturing at what time and what motion during the GAIT assessment the patient is experiencing pain, using a patient operated device. This information can be used to generate metrics about treatment progress and patient condition, as well as to generate concise evaluation reports that can be provided to physicians, patients to improve communication and patient treatment process. In addition, the collected data can be used as evidence if a patient does or does not have a medical condition, including possible uses relating to legal cases or insurance fraud, as well as to assess patients with fall risk and concussions.

Contrary to existing practices involving observational based assessment by orthopedists or other doctors, expensive joint and motion capturing setups, and subjective evaluation by a physician without any recorded data on the patient, the PT-ROME-GAIT system records the action of the joints simultaneously, providing objective data for diagnosis and evaluation. The motion capture setups are typically prohibitively expensive and require good lighting, and often involve indicators being placed on the patient's body to track points exactly. PT-ROME-GAIT uses a low-cost tracking sensor, which uses marker-less technology to track the specific points on the person being observed. The following flowcharts depict the processes described here.

Measuring Balance with Modified Ctsib and Functional Tests

The Balance measuring system for CTSIB comprises a ROME sensor, a remote device (C1) to access the software. The button BT1 on C1 is used to select the patient (who is the closest to the camera) on the screen MRK1. The therapist later selects the test to be performed. Therapist selects the section of modified CTSIB on MRK2 by using BTI to scroll and BT2 to select. The same setup is used to measure the patient's functional capability.

### Assessing Fall Risks of Inpatients in Hospital/Nursing Home:

A single or multiple ROME sensors are installed in the patients room, the system is connected to computer (CP2) that sends messages over cloud or network to server (CS1) about the status of the patient. When alerts are triggered messages are sent to smart phones (SP1), pagers (PG1) and other devices accessed by healthcare providers.

### Monitoring ADL:

ROME sensors are installed in one or multiple rooms, each system is connected to a computer (CP2) which is connected to a modem that is networked to a cloud server (CS2). The server transmits the results RS1, RS2, RS3 and RS4 the patients care provider or family.

### EXAMPLES

### ROME-Industrial Rehab and Ergonomics:

Three (3) example programs are provided to illustrate how the ROME system can be used for Industrial Rehab and Ergonomics.

### Example #1

The first program is a task trainer (T1) to provide feedback to a person when they perform a task in a non-ergonomic fashion. The person operates in the visible space of the sensor, and based on the information provided about the skeleton, their actions can be monitored. The task this system currently performs is verifying that employees can bend over and pick a large or heavy object up off of the ground without placing excessive stress on their spine. Using points M0, M1 and M2, an angle for spine bend is calculated (S2 and S3), and using information about M12, M13, M14, M16, M17 and M18 the bend of the knees can be tracked (S1). As the person performs a task, if these metrics deviate substantially from the accepted practice (i.e. do not bend forward more than 20 degrees), a warning message (W1) is shown to indicate that the person is doing something improperly. Through repeated training, the person should be able to stay within the acceptable limits, therefore improving how ergonomically they are performing the task.

### Example #2

The second program is a task efficiency measuring program (T2), designed to monitor how fast and accurately a person can perform a repetitive task. For this specific program, it measures how fast an employee can move a bar from below their waist to above their head, back to below their waist in a finite period of time. The idea here is if a person has to take something off a high or low shelf repeatedly, they need to actually be able to perform that action rapidly. By using the information from the sensor about M7 and M11, the actions of the person can be derived into metrics. Metrics are derived from observing the person, such as which zone they are in (S4), minimum and maximum travel of their hands (S5) and number of successful repetitions (S6). With this program, a manufacturer would have a specified minimum number of actions that must be accomplished in the timeframe. If the person did not meet or exceed the necessary number of actions, they would not be eligible for employment or be qualified to return to work.

### Example #3

The third program is a sterile area monitoring program (T3), designed to notify any person when they breech an area that has been designated as a sterile area. For this program, an area is indicated and set to be sterile (A1), and any time a tracked person enters the area a warning sound is played. The goal of this program is to raise awareness of people that they are interacting in a region and possibly affecting tools or sterility of devices contained therein.

### PT-ROME

With PT-ROME, a patient is going to perform specific actions to test how far they can move parts of their body, to track treatment progress of physical therapy. PT-ROME will have the therapist log in and enter the patient info if necessary, and allow the therapist to review past sessions of the patient through a recorded skeleton playback system (PB1). Once the therapist is caught up on the state of the patient, they can select which Range of Motion actions for the patient to perform (E1). The patient is given a pain indicator device (C1) and instructed to press the buttons (B1) when they encounter pain when performing the actions. The screen displays a virtual avatar of a person performing the desired action (ACT1), and the calculated bend angle is plotted on screen (GR1) along with any pain events (PE1) from the patient pressing the pain indicator button (B1). If a patient indicates pain during a test, a pain input screen (PE2) is shown after completing the current examination. This captures how intense the pain of the patient is on a standard pain scale. After completing all of the selected evaluations, a report is generated capturing things like maximum angle reached, intensity of pain and other parameters to aid the physical therapist. With this information, they can make treatment decisions and proceed with the rehabilitation exercises for the patient.

When a gait analysis needs to be performed, a patient would be placed in front of the PT-ROME-GAIT system and instructed in the actions they should perform. While performing the gait analysis actions (normally standing or running on a treadmill), the software will record the position and movement of the tracked points in 3D. After enough data has been captured, a trained professional can review the videos and determine the course of action to follow. Later in the development, automated algorithms can be designed to calculate and check for basic problems and offer easier assessment for the professional.

Measuring Balance with Modified CTSIB and Functional Tests:

With ROME for measuring balance and function, multiple tests are performed that include modified CTSIB, single leg stance test, Timed-Up-Go (TUG), functional reach. The system also performs an interactive training process to improve the balance of the patient. ROME for Balance will have the therapist log in and enter the patient info if necessary, and allow the therapist to review past sessions.

The therapist first marks the patient using the computer or a remote device (C1). Then the therapist instructs the patient to stand still with his legs together and hands on the side and selects among the four different CTSIB by clicking on BT1 on device (C1) to scroll between, eyes open on floor, eyes closed on floor, eyes open on foam and eyes closed on foam as shown in (MRK2). By clicking on (BT2) of device (C1) the therapist selects the condition he wants to administer. ROME sensor tracks the sway of the center of the body of the patient along X, Y and Z axes and is displayed as a live radial plot as in MRk3. The sensor also auto-detects when the patient moves his feet or hands to detect an event of loss of balance and records it as a fall as in (MRK4). ROME for balance software uses the cumulative sway for a period a time, average sway, average velocity of sway, peak velocity of sway, average acceleration and peak acceleration, range of sway along X, Y and Z dimensions and upper and lower body motion to determine a normative score for each test condition for CTSIB. Later that data is segregated into the visual, vestibular and somatosensory components as in (MRK7) of the balance by using the four different conditions data. An overall score is computed for every visit as in (MRK8) and the sway of the center of the body is reported as in (MRK9).

The test is administered to measure a baseline scenario and compare against population norms, the evaluation test can be repeated along the course of the treatment to measure improvement in patient's condition.

For the TUG test, the patient is similarly marked and the time taken by the patient to get up from a chair, walk 10 feet and comeback and sit is automatically computed. When a patient performs a TUG test, ROME for Balance automatically tracks the movement of all the limbs on the patient, measures center of body before and while getting up from the chair, measures the sway, velocity and acceleration of the patient. System also tracks the spine, hand movement, upper-lower body coordination and head position of the patient in 3D to determine their strategy while getting up from the chair. ROME for Balance also tracks the sway of the patient after they leave the chair support. When the patient is walking during TUG test Rome for Balance estimates total number of steps, stride length, height of each step, symmetry of placing legs, pain level of the patient, leg movement while turning 180 degrees, sway, total time taken, walking stance and step continuity real-time. These metrics are scored on a scale of 0 to 100 and ranked against population norms for the respective age group, sex, conditions and treatment.

Functional reach tests that are intended to assess dynamic balance are administered using ROME. This test measures the maximum distance a patient can reach forward beyond the arm's length while maintaining both feet on the ground in a standing position. The therapist first marks the patient using the computer or a remote device (C1). Then the therapist instructs the patient to stand still with his legs together and hands extended forward, and clicks on (BT2) on device (C1) to start the recording process. Rome for Balance automatically tracks and measures the location of the both wrists (M10 and M6) of the patient and measures the movement of the wrists when the patient reaches for the maximum distance and also measures the time it takes to reach that distance. If a patient moves during the test, the recording stops and marks it as an unsuccessful effort. Rome for Balance automatically adjusts for limitations in shoulder flexion to record an accurate measure of forward excursion. The measurement of forward excursion is made in real-world coordinates in meters and generates a report as in (MRK10). This is data is compared against a know set of parameters for particular age groups, sex and disease conditions and tracked over a period of time to measure progress in reach.

Single leg stance test is administered in a similar fashion by tracking patient's center of body and time a patient can perform the test when he is on right leg alone, left leg alone, legs in tandem and legs side by side.

Training a patient to improve his balance is an important functionality that would aid in reducing falls. Rome for Balance also provides a training module which would automatically train the patient to improve his balance. The patient is asked to move his body to reach a red target without moving his feat, ROME software calculates the time and the total distance the patient takes to reach a target. The software visually shows real-time how close a patient's center of body is to the target as in (MRK6). When the patient reaches a target a new target is displayed on the screen, all the movements and the patient's ability to move and maintain balance are tested under various scenarios with foam in some cases. A log of the results in maintained and compared against the norms and over a period of time with the same patient.

### Assessing Fall Risks of Inpatient in Hospital/Nursing Home:

Multiple ROME sensors are installed in the hospital rooms to track the movement of the patient. A patient is marked initially by the nurse as mentioned in Measuring balance with modified CTSIB and functional tests. Nurse also sets the thresholds for alert situations for a particular patient. ROME sensor keeps track of 20 joints of the patient to determine the condition and if the patient moves to get out of the bed, walk on the floor or if the patient is on the floor alerts are generated and sent to a central server in the hospital network or an the cloud from the computer connected to the sensor which monitoring the patient 24×7. The server based on the alert settings set by the healthcare provider for a particular patient will send the alert messages to a cell phone or pager pertaining to the room number where the patient is and the kind of alert triggered. ROME automatically recognizes if help is being offered to the patient and registers the help being offered to the patient.

### Monitoring ADL for Seniors in Home Settings:

ROME sensors are installed in multiple rooms where the senior lives. Setup and alerts are made in similar to the process in Assessing fall risks of inpatient in hospital/nursing home. In addition alerts are made when the senior performs any motion including getting up from chair, walking, taking pills, interacting with people, using the restroom, interactions with people. Reports are generated for ADL like how many times the senior got up from the chair, how much time the senior spent at a particular location, and how often the resident went between the living room, kitchen and bedroom. ROME for ADL can also detect multiple people in the room and can track the frequency and length of visits made by care giving personnel. The data is uploaded to a cloud and saved in a secure 128 bit encrypted server. The relatives of the senior can download an app on their smart phone that displays the level of activity. For assisted care and nursing home residents, the frequency and time of visits their loved ones are receiving at the nursing homes can be monitored through the app. The results look like in (MRK12) (RES1 to 4).

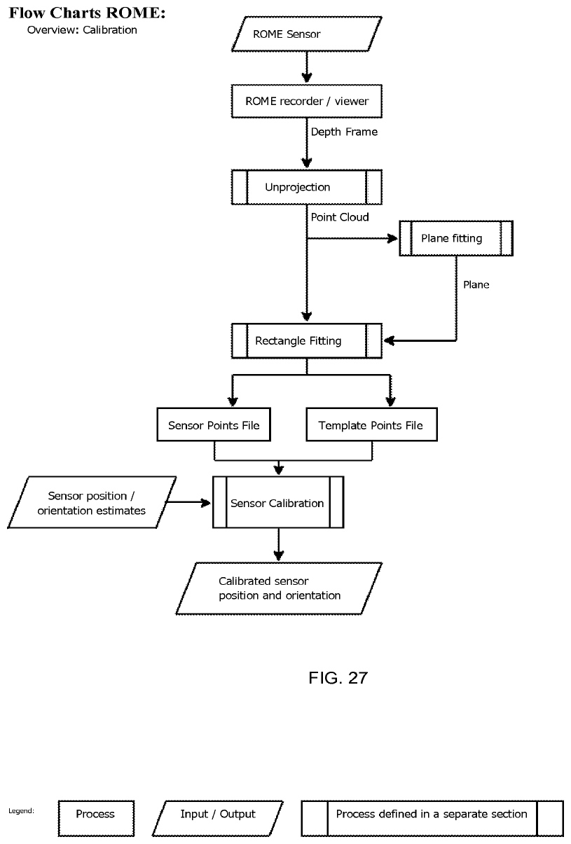

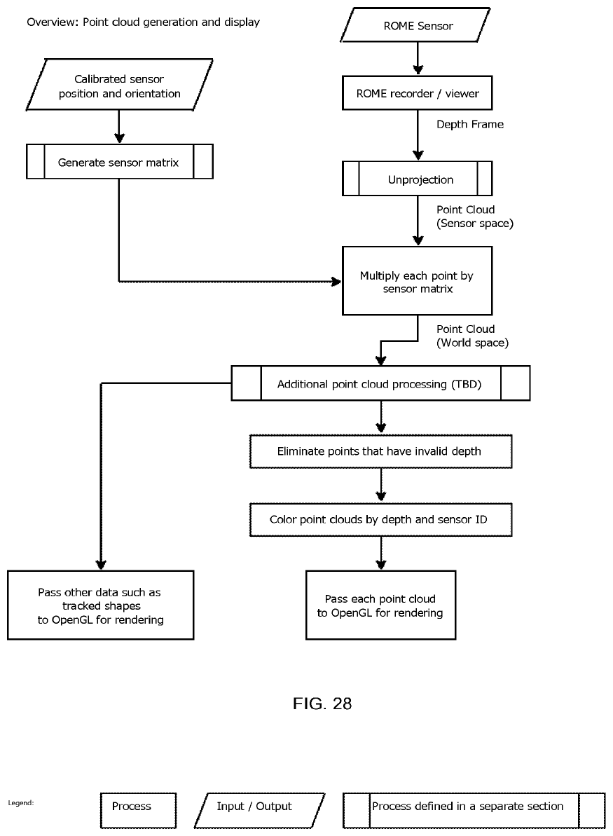

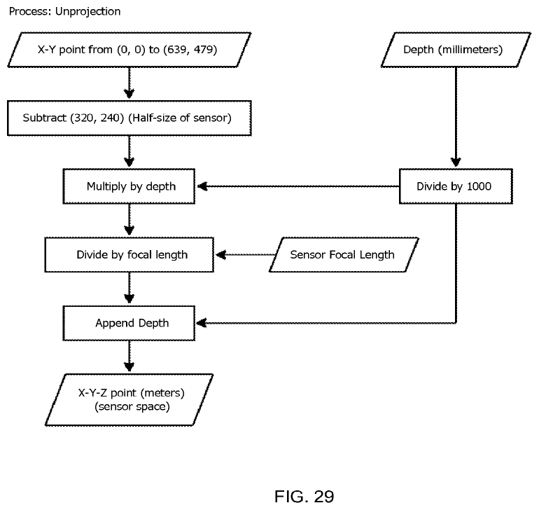

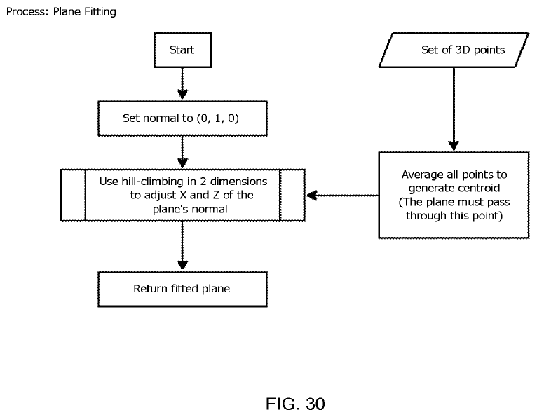

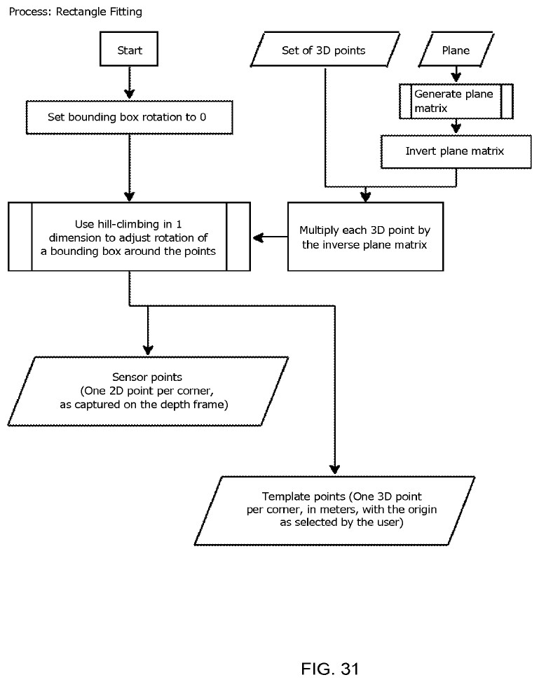

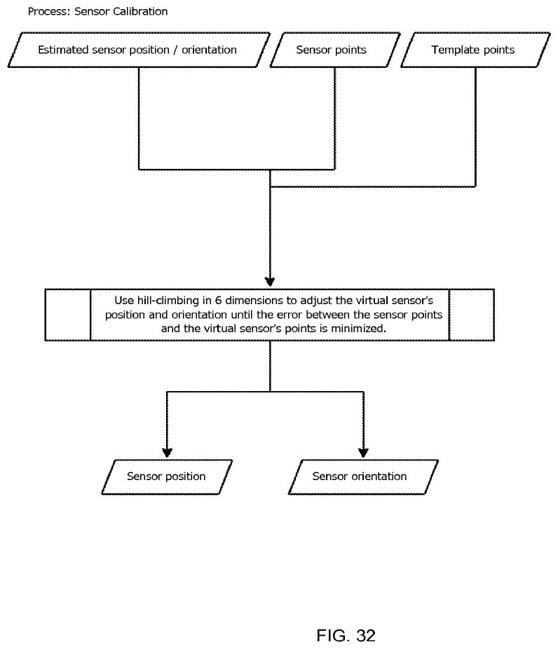

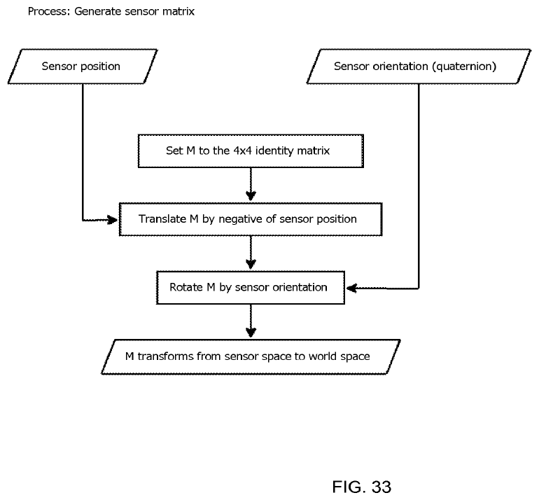

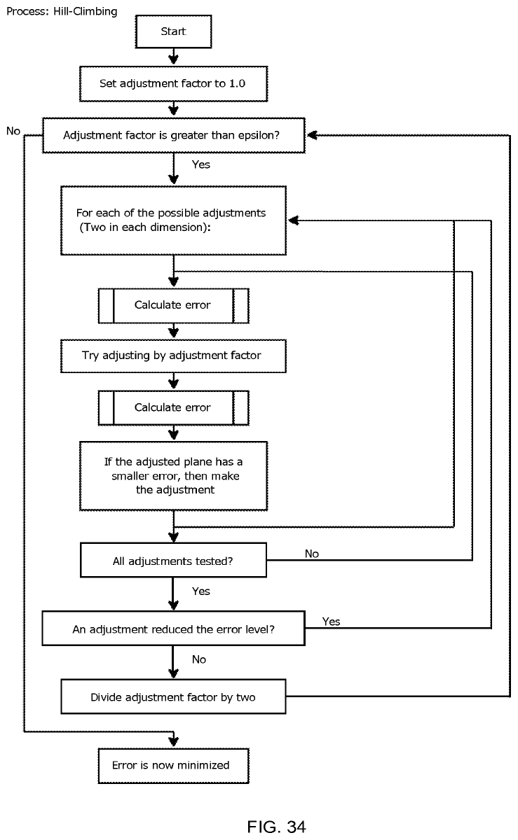

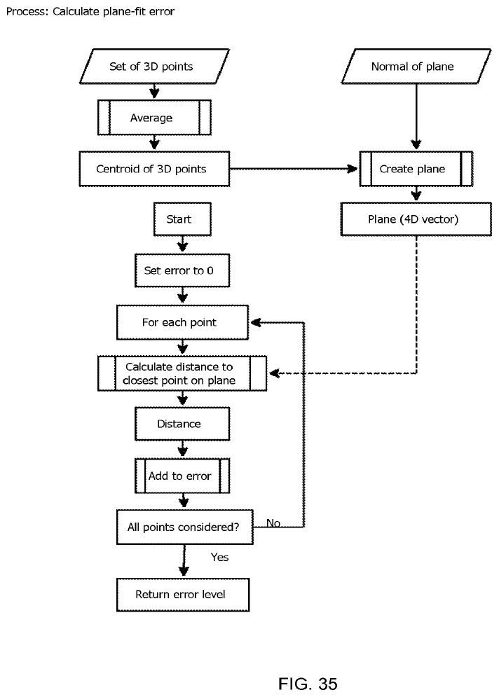

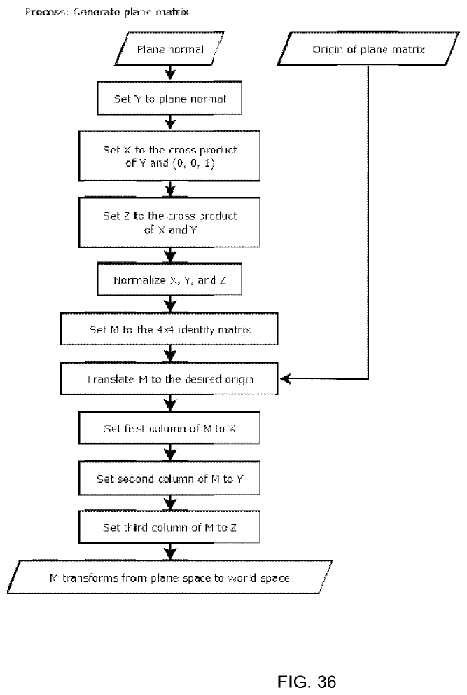

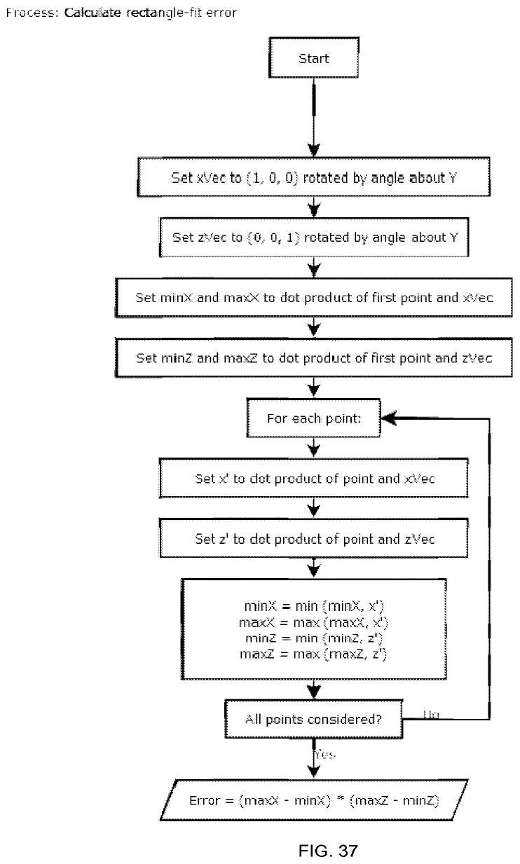

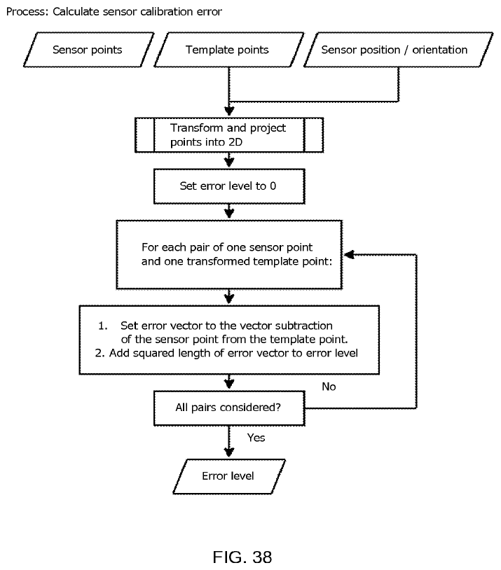

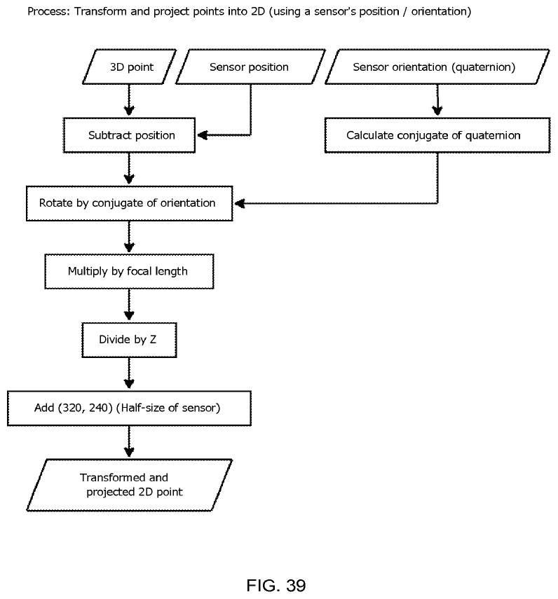

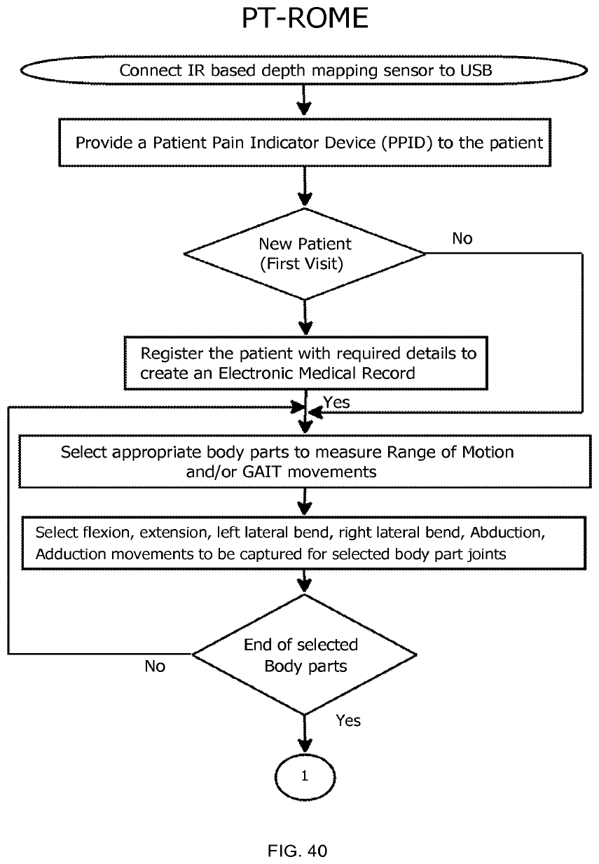

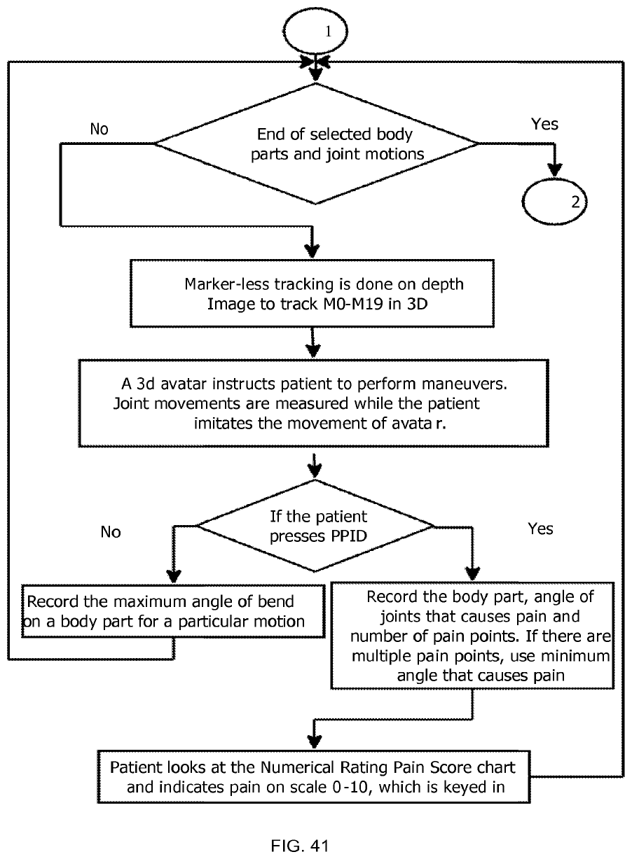

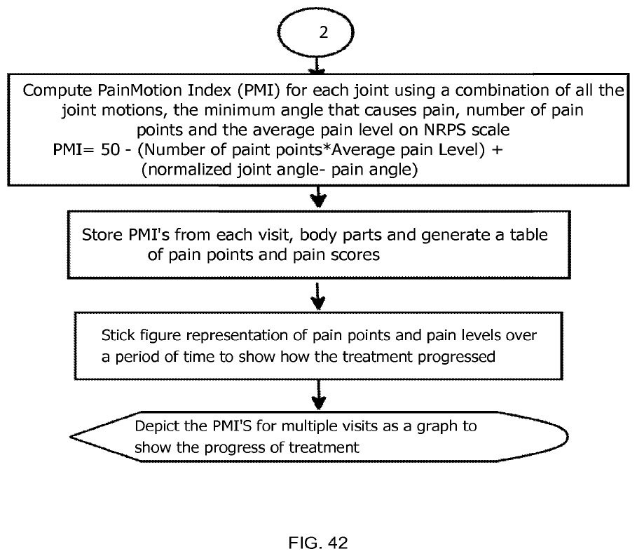

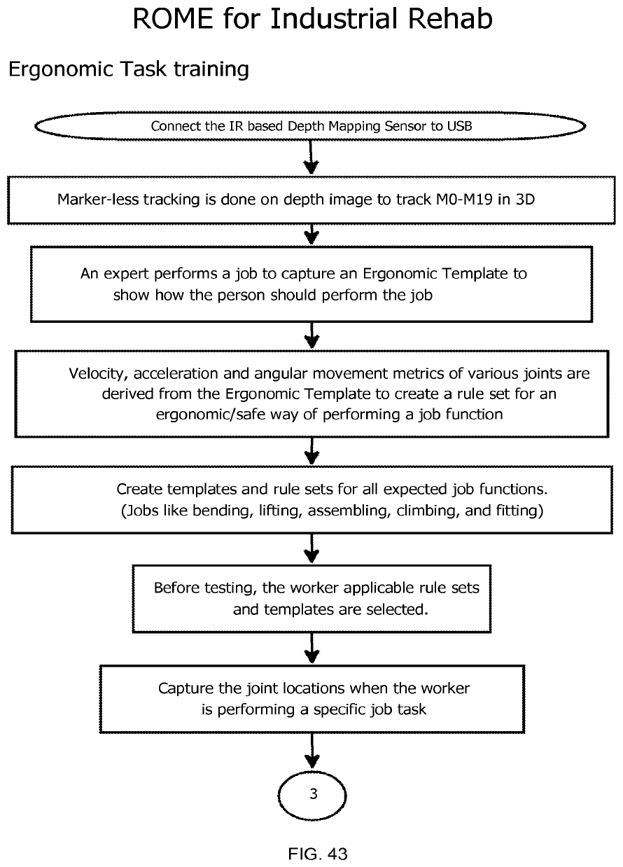

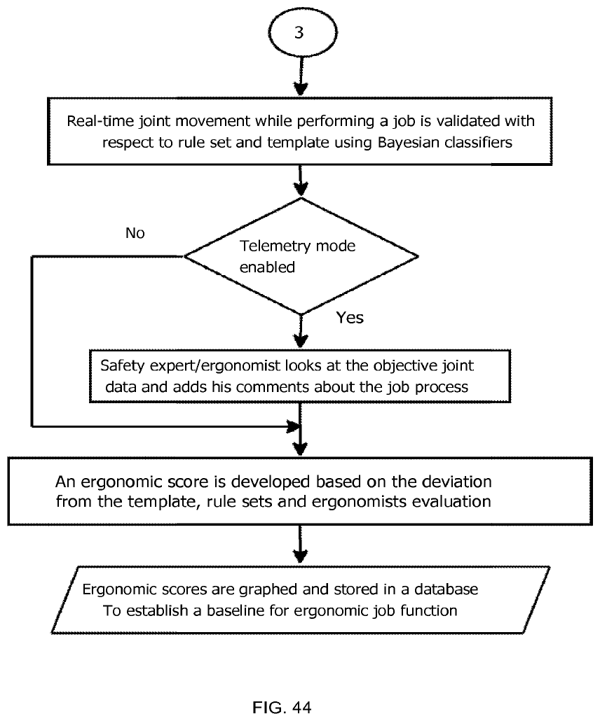

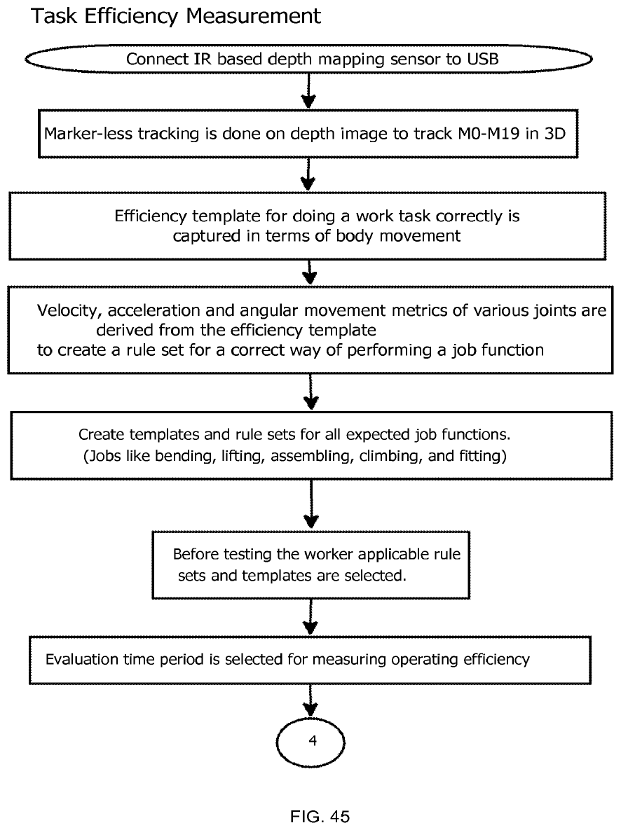

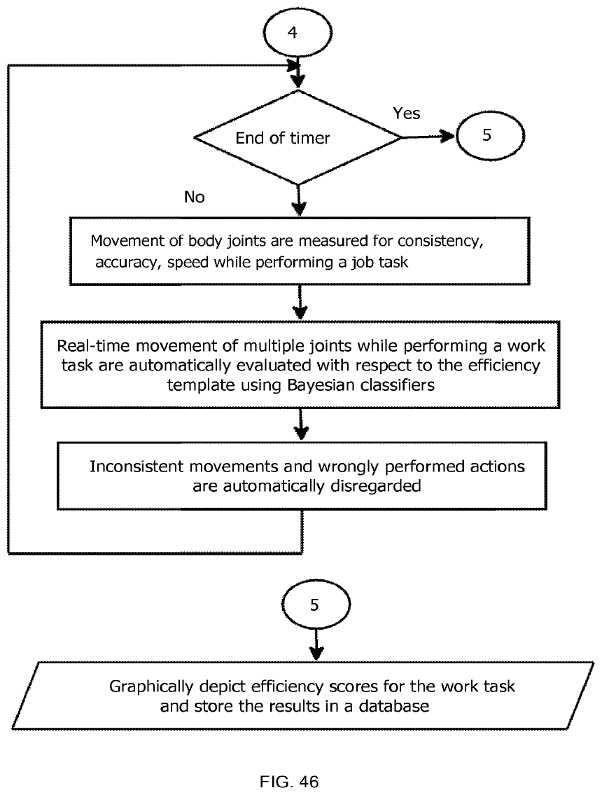

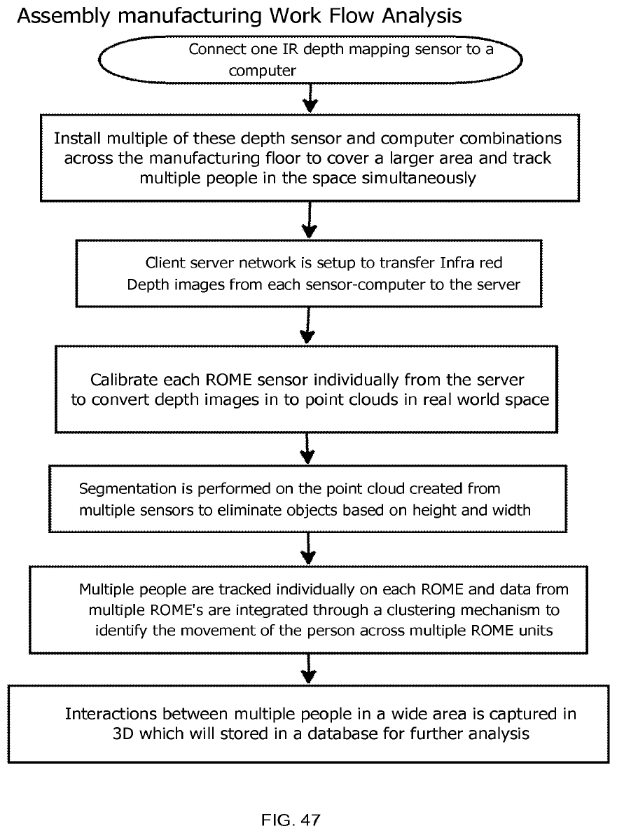

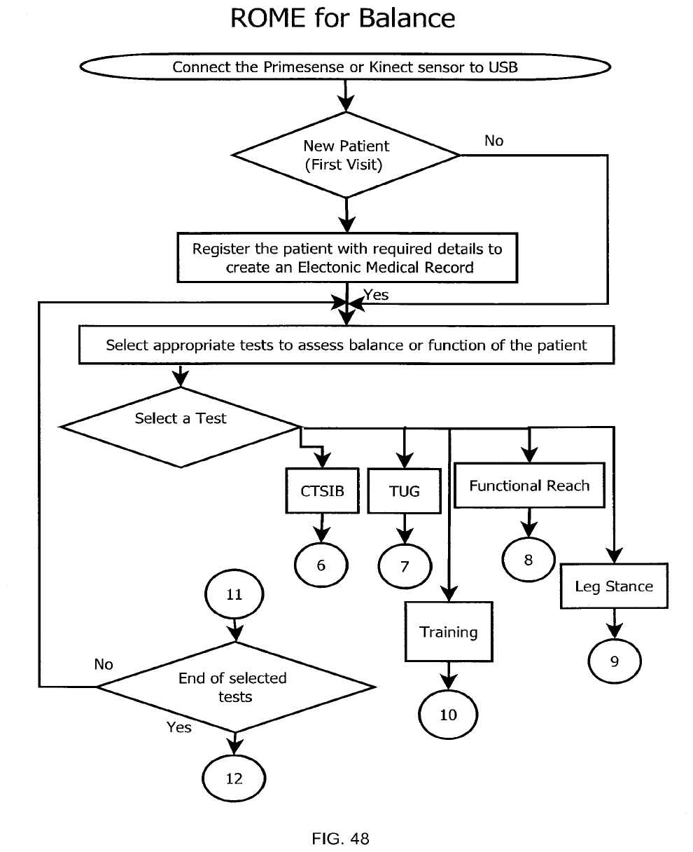

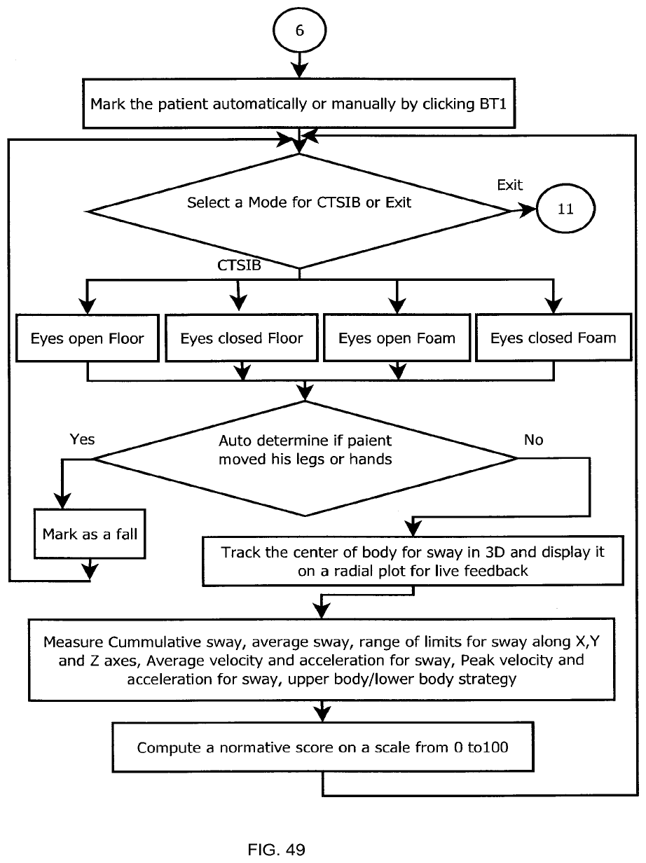

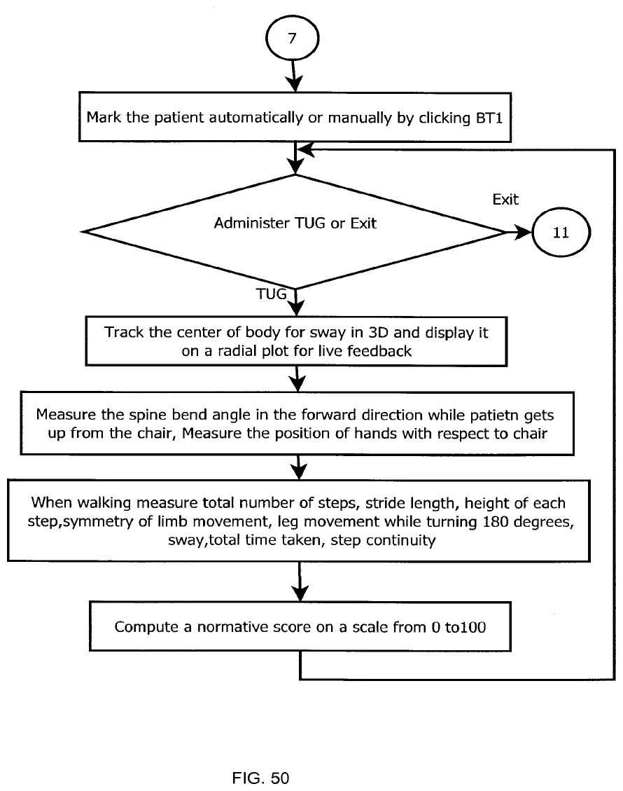

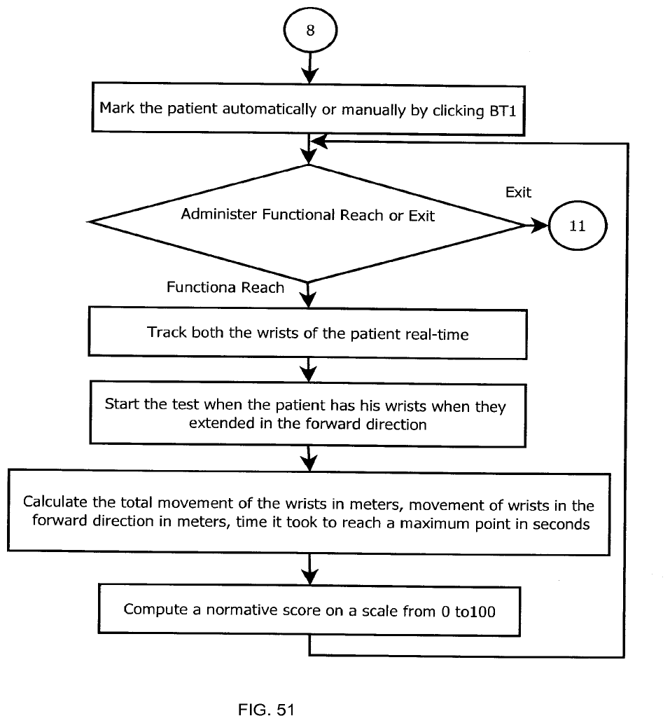

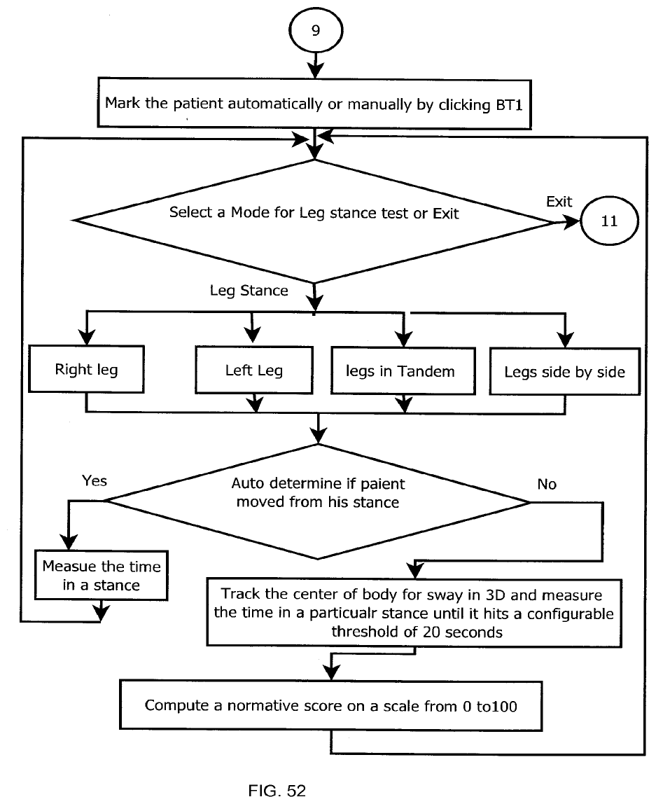

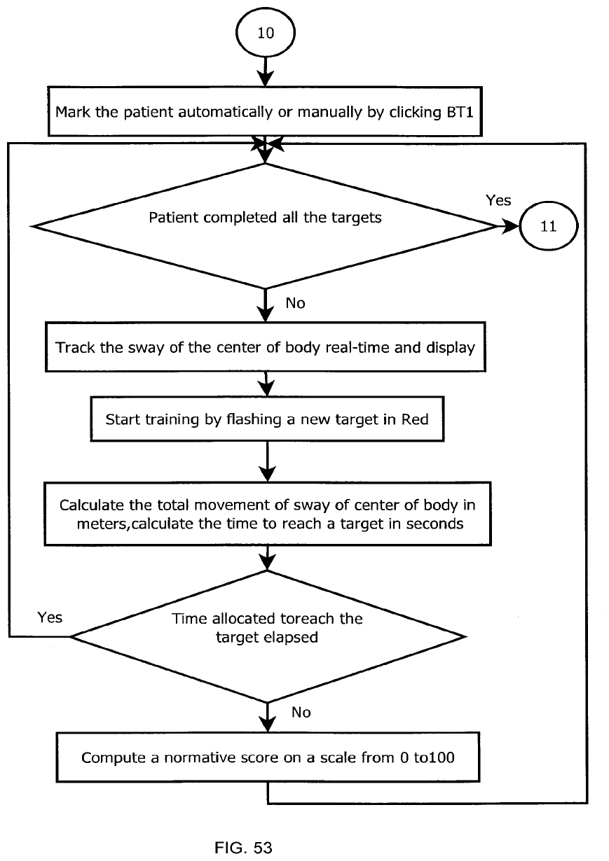

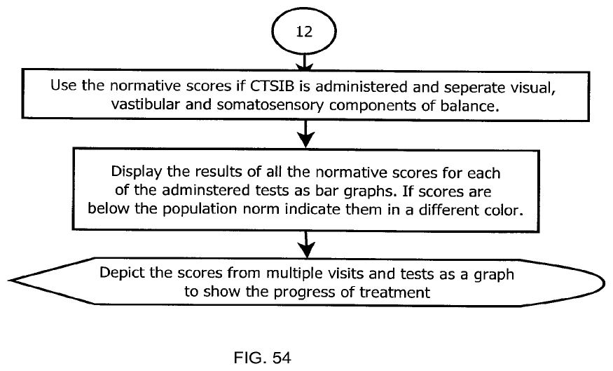

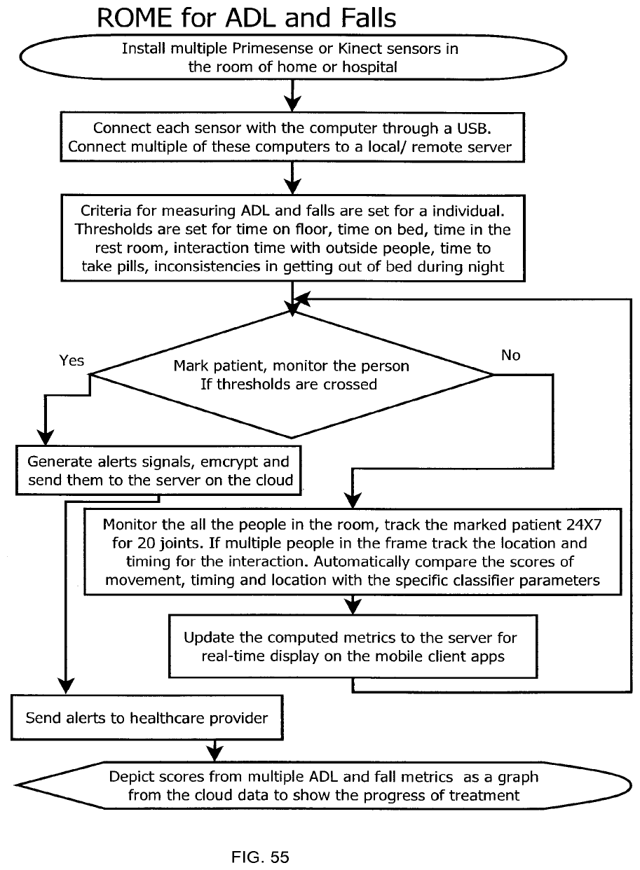

The flow charts for the various ROME systems and methods are shown in FIGS. 27-55.

The specific devices, systems, and methods described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

As used herein and in the appended claims, the singular forms “a,” “an,” and “the” include plural reference unless the context clearly dictates otherwise. Under no circumstances may the patent application be interpreted to be limited to the specific examples or embodiments or methods specifically disclosed herein.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Although the present invention has been specifically disclosed by preferred embodiments and optional features, modification and variation of the concepts herein disclosed may be resorted to by those skilled in the art, and such modifications and variations are considered to be within the scope of this invention as defined by the appended claims. In addition, the invention has been described broadly and generically herein. Each of the narrower species and subgeneric groupings falling within the generic disclosure also form part of the invention.

What is claimed is:

1. A range of motion evaluation system for monitoring and evaluating motion of a subject in real time, comprising:
a depth image based motion sensor configured to track motion of a subject in three dimensions and output a real-time motion signal corresponding thereto;
an electronic database storing a plurality of predefined motion patterns;
a processor configured to receive said real-time motion signal and to compare detected motion of said subject indicated by said real-time motion signal with predefined motion patterns stored in said database, said processor further being configured to generate an output signal indicating that said detected motion corresponds to a specific one or more of said plurality of predefined motion patterns; and
a monitoring device configured to receive said output signal and communicate a condition of said subject corresponding to the predefined motion pattern indicated by said output signal.

2. The range of motion evaluation system as set forth in claim 1, wherein said condition of said subject indicates that said subject is at risk for imminent injury.

3. The range of motion evaluation system as set forth in claim 2, wherein said subject is a hospital patient or a community dwelling person and said imminent injury is a fall.

4. The range of motion evaluation system as set forth in claim 2, wherein said subject is a hospital or nursing home patient and said imminent injury is bed or chair exit that could lead to a fall.

5. The range of motion evaluation system as set forth in claim 2, wherein said subject is a worker and said imminent injury is a workplace-related injury.

6. The range of motion evaluation system as set forth in claim 1, wherein said subject is a worker in a workplace and said condition of said subject indicates a measure of work performance of said subject.

7. The range of motion evaluation system as set forth in claim 6, wherein said measure of work performance is stored in a storage medium in a location associated with said subject.

8. The range of motion evaluation system as set forth in claim 1, wherein said subject is a homebound resident and said condition of said subject indicates a specific activity of said subject.

9. The range of motion evaluation system as set forth in claim 1, wherein said monitoring device is remotely located from said motion sensor.

10. The range of motion evaluation system as set forth in claim 9, wherein said processor communicates with said motion sensor over a communications network.

11. The range of motion evaluation system as set forth in claim 10, wherein said communications network is the Internet.

12. The range of motion evaluation system as set forth in claim 10, wherein said communications network is a local area network.

13. The range of motion evaluation system as set forth in claim 1, wherein said motion sensor is a marker-less sensor.

14. The range of motion evaluation system as set forth in claim 13, wherein marker-less sensor is an infrared depth sensor.

15. The range of motion evaluation system as set forth in claim 1, wherein said subject is a physical therapy/neuro/ortho patient and said condition of said subject indicates an amount of pain experienced by said subject at a specific point of motion as indicated by a pain indication device operated by said subject.

16. The range of motion evaluation system as set forth in claim 1, wherein said subject is a physical therapy patient and said condition of said subject indicates a gait of the subject.

17. The range of motion evaluation system as set forth in claim 1, wherein said subject is a physical therapy patient and said condition of said subject indicates a balance of the subject.

18. A computer-implemented method for monitoring and evaluating motion of a subject in real time, comprising:
tracking motion of a subject in three dimensions using a depth image based motion sensor and outputting a real-time motion signal corresponding thereto;
receiving, at a processor, said real-time motion signal and to compare detected motion of said subject indicated by said real-time motion signal with predefined motion patterns stored in an electronic database storing a plurality of predefined motion patterns, and generating an output signal indicating that said detected motion corresponds to a specific one or more of said plurality of predefined motion patterns; and
receiving at a monitoring device said output signal and communicating a condition of said subject corresponding to the predefined motion pattern indicated by said output signal.
